(19) United States
(12) Patent Application Publication
(10) Pub. No.: US 2018/0214133 A1
(43) Pub. Date: Aug. 2, 2018

(54) ULTRASONIC DIAGNOSTIC APPARATUS AND ULTRASONIC DIAGNOSTIC ASSISTANCE METHOD

(71) Applicant: Canon Medical Systems Corporation, Otawara-shi (JP)

(72) Inventors: Yoshitaka MINE, Nasushiobara (JP); Satoshi MATSUNAGA, Nasushiobara (JP); Yukifumi KOBAYASHI, Yokohama (JP); Kazuo TEZUKA, Nasushiobara (JP); Jiro HIGUCHI, Otawara (JP); Atsushi NAKAI, Nasushiobara (JP); Shigemitsu NAKAYA, Nasushiobara (JP); Yutaka KOBAYASHI, Nasushiobara (JP)

(73) Assignee: Canon Medical Systems Corporation, Otawara-shi (JP)

(21) Appl. No.: 15/883,219

(22) Filed: Jan. 30, 2018

(30) Foreign Application Priority Data
Jan. 31, 2017 (JP) 2017-015787

(51) Int. Cl. A61B 8/08; A61B 8/14

(52) U.S. Cl. CPC A61B 8/5223; A61B 8/463; A61B 8/5207; A61B 8/14

(57) ABSTRACT

According to one embodiment, an ultrasonic diagnostic apparatus includes processing circuitry. The processing circuitry is configured to set a plurality of small regions in at least one of a plurality of medical image data. The processing circuitry is configured to calculate a feature value of pixel value distribution of each small region. The processing circuitry is configured to generate a feature value image of the at least one of the plurality of medical image by using the calculated feature value. The processing circuitry is configured to execute an image registration between the plurality of medical image data by utilizing the feature value image.

### CROSS-REFERENCE TO RELATED APPLICATIONS

This application is based upon and claims the benefit of priority from the prior Japanese Patent Application No. 2017-015787, filed Jan. 31, 2017, the entire contents of all of which are incorporated herein by reference.

### FIELD

Embodiments described herein relate generally to an ultrasonic diagnostic apparatus and an ultrasonic diagnostic assistance method.

### BACKGROUND

In recent years, in medical image diagnosis, image registration between three-dimensional (3D) image data, which are acquired by using various medical image diagnostic apparatuses (an X-ray computer tomography apparatus, a magnetic resonance imaging apparatus, an ultrasonic diagnostic apparatus, an X-ray diagnostic apparatus, a nuclear medical diagnostic apparatus, etc.), has been performed by using various methods.

For example, image registration between 3D ultrasonic image data and 3D medical image data, such as an ultrasonic image, a CT (Computed Tomography) image, or an MR (magnetic resonance) image, which was acquired by using a medical image diagnostic apparatus in the past, is executed by acquiring, with use of an ultrasonic probe to which a position sensor is attached, 3D image data to which position information is added, and by using this position information and position information which is added to the other 3D medical image data.

### BRIEF DESCRIPTION OF THE DRAWINGS

FIG. 1 is a block diagram illustrating an ultrasonic diagnostic apparatus according to a first embodiment.

FIG. 2 is a flowchart illustrating an image registration process between ultrasonic image data according to the first embodiment.

FIG. 3 is a view illustrating an example of a case in which displacement between the ultrasonic image data is large.

FIG. 4 is a view illustrating an example of a case in which displacement between MR image data and ultrasonic image data is large.

FIG. 5 is a view illustrating a specific example of a feature value calculation process.

FIG. 6 is a view illustrating an example of a method of setting small regions.

FIG. 7 is a view illustrating an example of a feature value image.

FIG. 8 is a view illustrating an example of a mask region.

FIG. 9 is a block diagram illustrating an ultrasonic diagnostic apparatus according to a second embodiment.

FIG. 10 is a flowchart illustrating a registration process between ultrasonic image data according to the second embodiment.

FIG. 11 is a flowchart illustrating a registration process in a case in which a displacement occurs.

FIG. 12 is a view illustrating an example of ultrasonic image display before registration between the ultrasonic image data after completion of sensor registration.

FIG. 13 is a view illustrating an example of ultrasonic image display after the registration between the ultrasonic image data.

FIG. 14 is a flowchart illustrating a registration process between ultrasonic image data and medical image data according to a third embodiment.

FIG. 15A is a conceptual view of sensor registration between ultrasonic image data and medical image data.

FIG. 15B is a conceptual view of sensor registration between ultrasonic image data and medical image data.

FIG. 15C is a conceptual view of sensor registration between ultrasonic image data and medical image data.

FIG. 16A is a view illustrating an example in which ultrasonic image data and medical image data are associated.

FIG. 16B is a view illustrating an example in which ultrasonic image data and medical image data are associated.

FIG. 17 is a view for describing correction of displacement between ultrasonic image data and medical image data.

FIG. 18 is a view illustrating an example of acquisition of ultrasonic image data in a state in which the correction of displacement is completed.

FIG. 19 is a view illustrating an example of ultrasonic image display after registration between ultrasonic image data and medical image data.

FIG. 20 is a view illustrating an example of synchronous display between an ultrasonic image and a medical image.

FIG. 21 is a view illustrating another example of synchronous display between an ultrasonic image and a medical image.

FIG. 22 is a block diagram illustrating an ultrasonic diagnostic apparatus in a case of utilizing infrared for a position sensor system.

FIG. 23 is a block diagram illustrating an ultrasonic diagnostic apparatus in a case of utilizing robotic arms for a position sensor system.

FIG. 24 is a block diagram illustrating an ultrasonic diagnostic apparatus in a case of utilizing a gyro sensor for a position sensor system.

FIG. 25 is a block diagram illustrating an ultrasonic diagnostic apparatus in a case of utilizing a camera for a position sensor system.

### DETAILED DESCRIPTION

There are the following problems in the image registration using the 3D ultrasonic image data by the conventional method.

In the conventional technique, there is image registration, by utilizing brightness information of an ultrasonic image, a CT image, or an MR image, using a mutual information, a correlation coefficient, a brightness difference, etc., and the registration is mostly executed, as regions in images for registration, between whole regions or main regions (e.g., ROI: Region of Interest) of the images. However, factors such as a speckle noise, an acoustic shadow, a multiple artifact, depth-dependent brightness attenuation, lowering of side brightness, brightness unevenness after STC (Sensitivity Time Control) adjustment inhibit improvement in registration precision of an ultrasonic image. In particular, a speckle noise obscuring structural information also becomes an inhibiting factor in registration.

In addition, since 3D ultrasonic image data is acquired from an arbitrary direction, the degree of freedom in an initial positional relationship between volume data for registration is large, which may result in difficulty in registration.

From the above points, even if the image registration which has been conventionally executed between CT images is applied to image registration including an ultrasonic image, the precision would still be low. Furthermore, the success rates of the image registration between 3D ultrasonic image data and 3D ultrasonic image data and the image registration between 3D ultrasonic image data and 3D medical image data by the conventional methods are low, and it can be said that the image registration between 3D ultrasonic image data and 3D ultrasonic image data and the image registration between 3D ultrasonic image data and 3D medical image data by the conventional methods are not practical.

In general, according to one embodiment, an ultrasonic diagnostic apparatus includes processing circuitry. The processing circuitry is configured to set a plurality of small regions in at least one of a plurality of medical image data. The processing circuitry is configured to calculate a feature value of pixel value distribution of each small region. The processing circuitry is configured to generate a feature value image of the at least one of the plurality of medical image by using the calculated feature value. The processing circuitry is configured to execute an image registration between the plurality of medical image data by utilizing the feature value image.

In the following descriptions, an ultrasonic diagnostic apparatus and an ultrasonic diagnostic assistance method according to 6—the present embodiments will be described with reference to the drawings. In the embodiments described below, elements assigned with the same reference symbols perform the same operations, and redundant descriptions thereof will be omitted as appropriate.

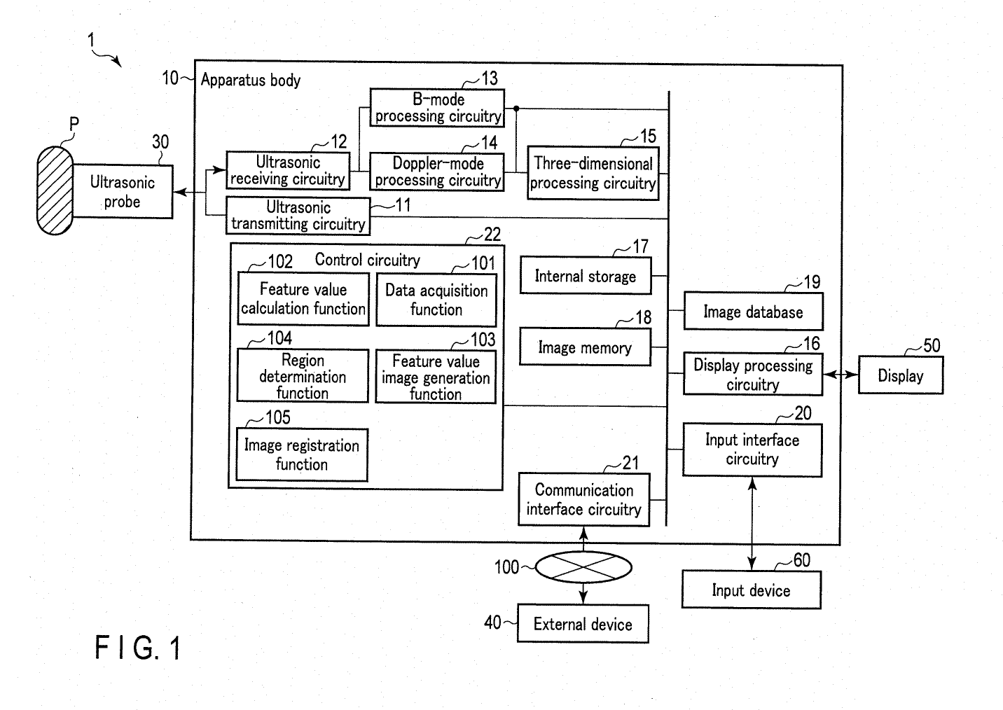

FIG. 1 is a block diagram illustrating a configuration example of an ultrasonic diagnostic apparatus 1 according to an embodiment. As illustrated in FIG. 1, the ultrasonic diagnostic apparatus 1 includes an apparatus body 10 and an ultrasonic probe 30. The apparatus body 10 is connected to an external device 40 via a network 100. In addition, the apparatus body 10 is connected to a display 50 and an input device 60.

The ultrasonic probe 30 includes a plurality of piezoelectric transducers, a matching layer provided on the piezoelectric transducers, and a backing material for preventing the ultrasonic waves from propagating backward from the piezoelectric transducers. The ultrasonic probe 30 is detachably connected to the apparatus body 10. Each of the plurality of piezoelectric transducers generates an ultrasonic wave based on a driving signal supplied from ultrasonic transmitting circuitry 11 included in the apparatus body 10. In addition, buttons, which are pressed at a time of an offset process, at a time of a freeze of an ultrasonic image, etc., may be disposed on the ultrasonic probe 30.

When the ultrasonic probe 30 transmits ultrasonic waves to a living body P, the transmitted ultrasonic waves are sequentially reflected by a discontinuity surface of acoustic impedance of the living tissue of the living body P, and received by the plurality of piezoelectric transducers of the ultrasonic probe 30 as a reflected wave signal. The amplitude of the received reflected wave signal depends on an acoustic impedance difference on the discontinuity surface by which the ultrasonic waves are reflected. Note that the frequency of the reflected wave signal generated when the transmitted ultrasonic pulses are reflected by moving blood or the surface of a cardiac wall, etc. shifts depending on the velocity component of the moving body in the ultrasonic transmission direction due to the Doppler effect. The ultrasonic probe 30 receives the reflected wave signal from the living body P, and converts it into an electrical signal.

The ultrasonic probe 30 according to the present embodiment is a one-dimensional array probe including a plurality of ultrasonic transducers which two-dimensionally scans the living body P. In the meantime, the ultrasonic probe 30 may be a mechanical four-dimensional probe (a three-dimensional probe of a mechanical swing method) which is configured such that a one-dimensional array probe and a motor for swinging the probe are provided in a certain enclosure, and ultrasonic transducers are swung at a predetermined angle (swing angle). Thereby, a tilt scan or rotational scan is mechanically performed, and the living body P is three-dimensionally scanned. Besides, the ultrasonic probe 30 may be a two-dimensional array probe in which a plurality of ultrasonic transducers are arranged in a matrix, or a 1.5-dimensional array probe in which a plurality of transducers that are one-dimensionally arranged are divided into plural parts.

The apparatus body 10 illustrated in FIG. 1 is an apparatus which generates an ultrasonic image, based on the reflected wave signal which the ultrasonic probe 30 receives. As illustrated in FIG. 1, the apparatus body 10 includes the ultrasonic transmitting circuitry 11, ultrasonic receiving circuitry 12, B-mode processing circuitry 13, Doppler-mode processing circuitry 14, three-dimensional processing circuitry 15, display processing circuitry 16, an internal storage 17, an image memory 18 (cine memory), an image database 19, input interface circuitry 20, communication interface circuitry 21, and control circuitry 22.

The ultrasonic transmitting circuitry 11 is a processor which supplies a driving signal to the ultrasonic probe 30. The ultrasonic transmitting circuitry 11 is realized by, for example, trigger generating circuitry, delay circuitry, and pulser circuitry. The trigger generating circuitry repeatedly generates, at a predetermined rate frequency, rate pulses for forming transmission ultrasonic. The delay circuitry imparts, to each rate pulse generated by the trigger generating circuitry, a delay time for each piezoelectric transducer which is necessary for determining transmission directivity by converging ultrasonic, which is generated from the ultrasonic probe 30, into a beam form. The pulser circuitry applies a driving signal (driving pulse) to the ultrasonic probe 30 at a timing based on the rate pulse. By varying the delay time that is imparted to each rate pulse by the delay circuitry, the transmission direction from the piezoelectric transducer surface can discretionarily be adjusted.

The ultrasonic receiving circuitry 12 is a processor which executes various processes on the reflected wave signal which the ultrasonic probe 30 receives, and generates a reception signal. The ultrasonic receiving circuitry 12 is realized by, for example, amplifier circuitry, an A/D converter, reception delay circuitry, and an adder. The amplifier circuitry executes a gain correction process by amplifying, on a channel-by-channel basis, the reflected wave signal which the ultrasonic probe 30 receives. The A/D converter converts the gain-corrected reflected wave signal to a digital signal. The reception delay circuitry imparts a delay time, which is necessary for determining reception directivity, to the digital signal. The adder adds a plurality of digital signals to which the delay time was imparted. By the addition process of the adder, a reception signal is generated in which a reflected component from a direction corresponding to the reception directivity is emphasized.

The B-mode processing circuitry 13 is a processor which generates B-mode data, based on the reception signal received from the ultrasonic receiving circuitry 12. The B-mode processing circuitry 13 executes an envelope detection process and a logarithmic amplification process on the reception signal received from the ultrasonic receiving circuitry 12, and generates data (hereinafter, B-mode data) in which the signal strength is expressed by the magnitude of brightness. The generated B-mode data is stored in a RAW data memory (not shown) as B-mode RAW data on an ultrasonic scanning line. The B-mode RAW data may be stored in the internal storage 17 (to be described later).

The Doppler-mode processing circuitry 14 is a processor which generates a Doppler waveform and Doppler data, based on the reception signal received from the ultrasonic receiving circuitry 12. The Doppler-mode processing circuitry 14 extracts a blood flow signal from the reception signal, generates a Doppler waveform from the extracted blood flow signal, and generates data (hereinafter, Doppler data) in which information, such as a mean velocity, variance and power, is extracted from the blood flow signal with respect to multiple points.

The three-dimensional processing circuitry 15 is a processor which can generate two-dimensional image data or three-dimensional image data (hereinafter, also referred to as “volume data”), based on the data generated by the B-mode processing circuitry 13 and the Doppler-mode processing circuitry 14. The three-dimensional processing circuitry 15 generates two-dimensional image data which is composed of pixels, by executing RAW-pixel conversion.

Furthermore, the three-dimensional processing circuitry 15 generates volume data which is composed of voxels in a desired range, by executing RAW-voxel conversion, which includes an interpolation process with spatial position information being taken into account, on the B-mode RAW data stored in the RAW data memory. The three-dimensional processing circuitry 15 generates rendering image data by applying a rendering process to the generated volume data. Hereinafter, the B-mode RAW data, two-dimensional image data, volume data, and rendering image data are also collectively called ultrasonic image data.

The display processing circuitry 16 executes various processes, such as dynamic range, brightness, contrast and y curve corrections, and RGB conversion, on various image data generated in the three-dimensional processing circuitry 15, thereby converting the image data to a video signal. The display processing circuitry 16 causes the display 50 to display the video signal. In the meantime, the display processing circuitry 16 may generate a user interface (GUI: Graphical User Interface) for an operator to input various instructions by the input interface circuitry 20, and may cause the display 50 to display the GUI. For example, a CRT display, a liquid crystal display, an organic EL display, an LED display, a plasma display, or other discretionary display known in the present technical field, may be used as needed as the display 50.

The internal storage 17 includes, for example, a storage medium which can be read by a processor, such as a magnetic or optical storage medium, or a semiconductor memory. The internal storage 17 stores a control program for realizing ultrasonic transmission/reception, a control program for executing an image process, and a control program for executing a display process. In addition, the internal storage 17 stores diagnosis information (e.g. patient ID, doctor's findings, etc.), a diagnosis protocol, a body mark generation program, and data such as a conversion table for presetting a range of color data for use in imaging, with respect to each of regions of diagnosis. Besides, the internal storage 17 may store anatomical illustrations, for example, an atlas, relating to the structures of internal organs in the body.

In addition, the internal storage 17 stores two-dimensional image data, volume data and rendering image data which were generated by the three-dimensional processing circuitry 15, in accordance with a storing operation which is input via the input interface circuitry 20. Furthermore, in accordance with a storing operation which is input via the input interface circuitry 20, the internal storage 17 may store two-dimensional image data, volume data and rendering image data which were generated by the three-dimensional processing circuitry 15, along with the order of operations and the times of operations. The internal storage 17 can transfer the stored data to an external device via the communication interface circuitry 21.

The image memory 18 includes, for example, a storage medium which can be read by a processor, such as a magnetic or optical storage medium, or a semiconductor memory. The image memory 18 stores image data corresponding to a plurality of frames immediately before a freeze operation which is input via the input interface circuitry 20. The image data stored in the image memory 18 is, for example, successively displayed (cine-displayed).

The image database 19 stores image data which is transferred from the external device 40. For example, the image database 19 receives past medical image data relating to the same patient, which was acquired in past diagnosis and is stored in the external device 40, and stores the past medical image data. The past medical image data includes ultrasonic image data, CT (Computed Tomography) image data, MR image data, PET (Positron Emission Tomography)-CT image data, PET-MR image data, and X-ray image data.

The image database 19 may store desired image data by reading in image data which is stored in storage media such as an MO, CD-R and DVD.

The input interface circuitry 20 accepts various instructions from the user via the input device 60. The input device 60 is, for example, a mouse, a keyboard, a panel switch, a slider switch, a trackball, a rotary encoder, an operation panel, and a touch command screen (TCS). The input interface circuitry 20 is connected to the control circuitry 22, for example, via a bus, converts an operation instruction, which is input from the operator, to an electric signal, and outputs the electric signal to the control circuitry 22. In the present specification, the input interface circuitry 20 is not limited to input interface which is connected to physical operation components such as a mouse and a keyboard. Examples of the input interface circuitry 20 include processing circuitry of an electric signal, which receives, as a wireless signal, an electric signal corresponding to an operation instruction that is input from an external input device provided separately from the ultrasonic diagnostic apparatus 1, and outputs this electric signal to the control circuitry 22. For example, the input interface circuitry 20 may be an external input device capable of transmitting, as a wireless signal, an operation instruction corresponding to an instruction by a gesture of an operator.

The communication interface circuitry 21 is connected to the external device 40 via the network 100, etc., and executes data communication with the external device 40. The external device 40 is, for example, a database of a PACS (Picture Archiving and Communication System) which is a system for managing the data of various kinds of medical images, or a database of an electronic medical record system for managing electronic medical records to which medical images are added. In addition, the external device 40 is, for example, various kinds of medical image diagnostic apparatuses other than the ultrasonic diagnostic apparatus 1 according to the present embodiment, such as an X-ray CT apparatus, an MRI (Magnetic Resonance Imaging) apparatus, a nuclear medical diagnostic apparatus, and an X-ray diagnostic apparatus. In the meantime, the standard of communication with the external device 40 may be any standard. An example of the standard is DICOM (digital imaging and communication in medicine).

The control circuitry 22 is, for example, a processor which functions as a central unit of the ultrasonic diagnostic apparatus 1. The control circuitry 22 executes a control program which is stored in the internal storage, thereby realizing functions corresponding to this program. Specifically, the control circuitry 22 executes a data acquisition function 101, a feature value calculation function 102, a feature value image generation function 103, a region determination function 104, and an image registration function 105.

By executing the data acquisition function 101, the control circuitry 22 acquires ultrasonic image data from the three-dimensional processing circuitry 15. In a case of acquiring B-mode RAW data as ultrasonic image data, the control circuitry 22 may acquire the B-mode RAW data from the B-mode processing circuitry 13.

By executing the feature value calculation function 102, the control circuitry 22 sets small regions in image data and extracts a feature value of pixel value distribution of each small region from medical image data. An example of a feature value of pixel value distribution of a small region is a feature value relating to pixel value variation of a small region. Variance and standard deviation of pixel values of a small region are examples. Another example of a feature value of pixel value distribution of a small region is a feature value relating to a primary differential of pixel values of the small region. A gradient vector and a gradient value are examples. A further example of a feature value of pixel value distribution of a small region is a feature value relating to a secondary differential of pixel values of a small region.

By executing the feature value image generation function 103, the control circuitry 22 generates a feature value image by using a feature value calculated from medical image data and ultrasonic image data.

By executing the region determination function 104, the control circuitry 22, for example, accepts an input from the user into the input device 60 via the input interface circuitry 20, and determines an initial positional relationship for registration between medical image data based on the input.

By executing the image registration function 105, the control circuitry 22 executes image registration based on the similarity between medical image data. In addition, in a case in which an initial positional relationship for registration between medical image data is determined, the control circuitry 22 may execute image registration by utilizing the determined initial positional relationship.

The feature value calculation function 102, feature value image generation function 103, region determination function 104, and image registration function 105 may be assembled as the control program. Alternatively, dedicated hardware circuitry, which can execute these functions, may be assembled in the control circuitry 22 itself, or may be assembled in the apparatus body 10.

The control circuitry 22 may be realized by an application-specific integrated circuit (ASIC) in which this dedicated hardware circuitry is assembled, a field programmable logic device (FPGA), a complex programmable logic device (CPLD), or a simple programmable logic device (SPLD).

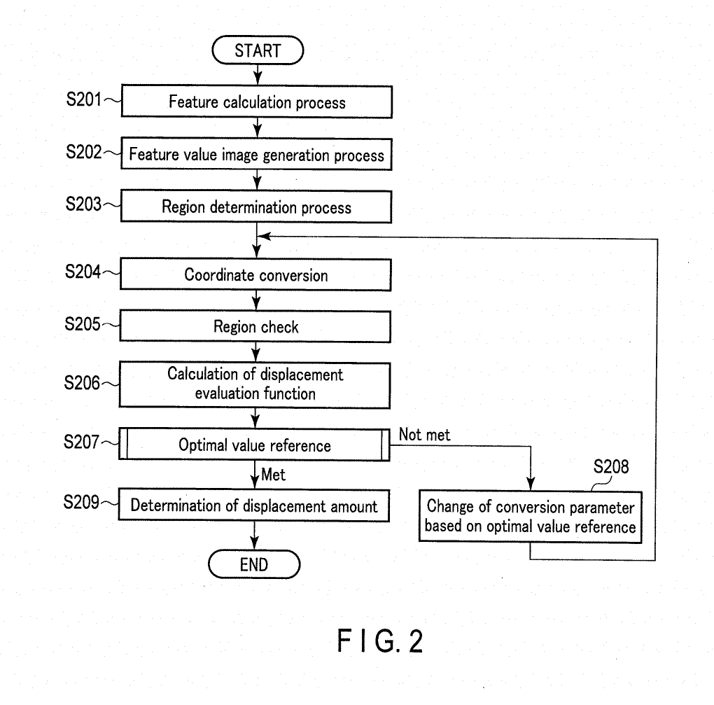

Next, image registration of the ultrasonic diagnostic apparatus 1 according to the first embodiment will be described with reference to the flowchart of FIG. 2. In the first embodiment to be described below, a case is assumed in which image registration between ultrasonic image data being imaged in a current examination and past ultrasonic image data of imaging an identical portion as medical image data to be an image registration target is executed. In addition, a case in which ultrasonic image data is volume data is assumed.

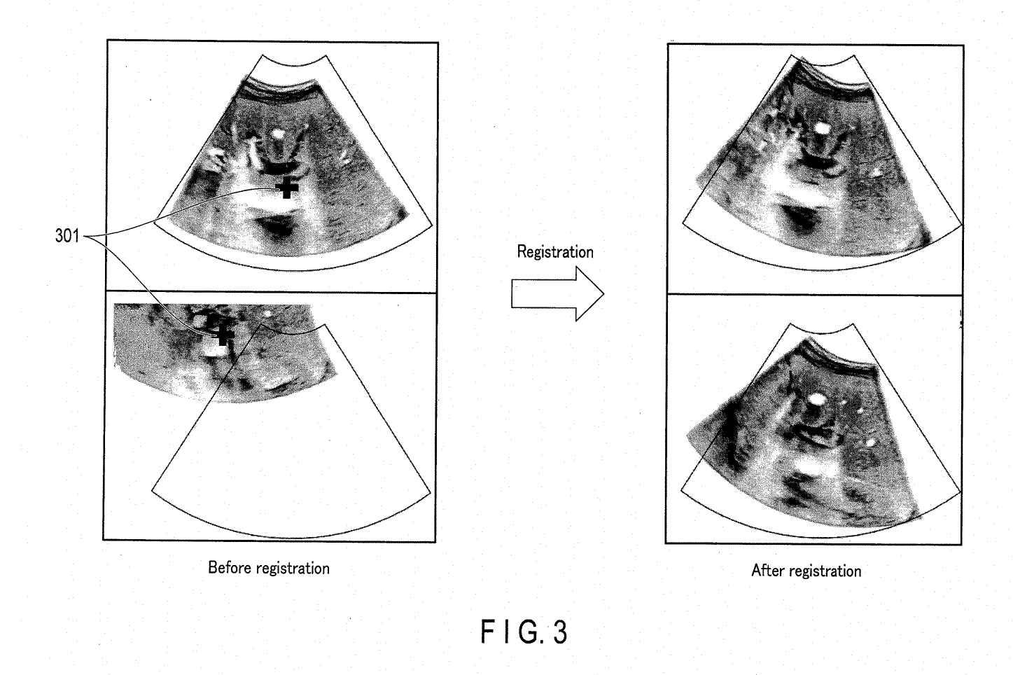

In step S201, the control circuitry 22, which executes the feature value calculation function 102, calculates a feature value relating to a variation in brightness as a pre-process for first volume data of the current ultrasonic image data and second volume data of the past medical image data. In the present embodiment, a value relating to a gradient value (primary differential) of a brightness value is used as a feature value. A method of calculating a feature value will be described later with reference to FIG. 3.

In step S202, the control circuitry 22, which executes the feature value image generation function 103, generates a first feature value image (also referred to as “first gradient value image”) based on a feature value of the first volume data and a second feature value image (also referred to as “second gradient value image”) based on a feature value of the second volume data.

In step S203, the control circuitry 22, which executes the region determination function 104, sets a mask region to be processed with respect to the first feature value image and the second feature value image. Furthermore, the control circuitry 22 determines an initial positional relationship for registration.

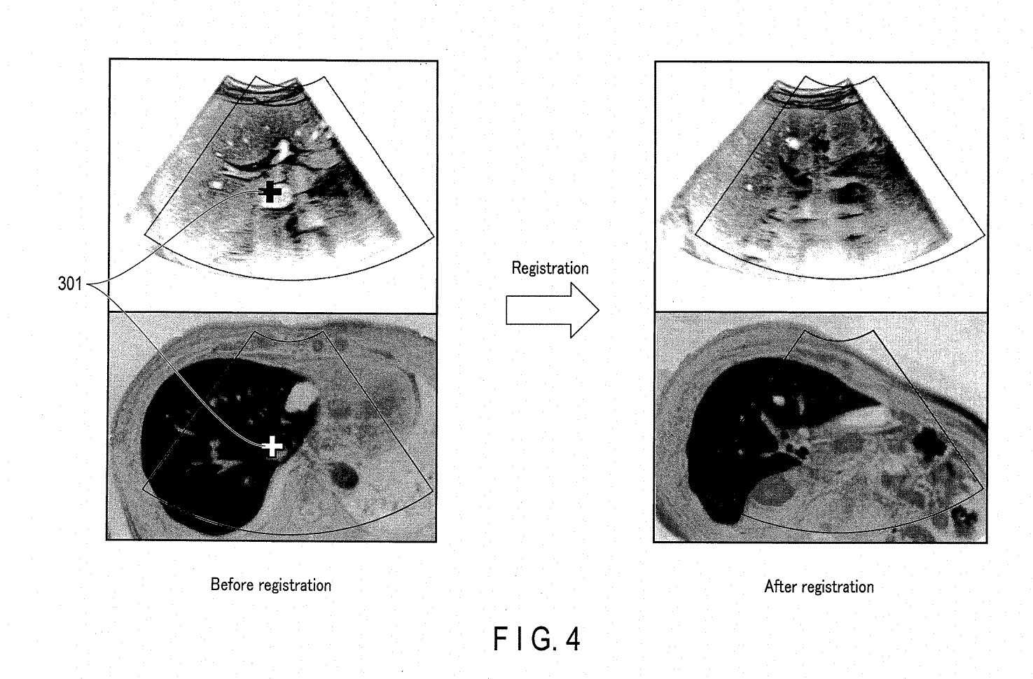

Herein, a method of determining an initial positional relationship for registration will be described with reference to FIGS. 3 and 4. FIG. 3 illustrates an example of a case in which displacement between ultrasonic image data is large, and FIG. 4 illustrates an example of a case in which displacement between MR image data and ultrasonic image data is large. As illustrated in FIGS. 3 and 4, as a method of determining an initial positional relationship for registration, a user clicking corresponding points 301 on the images is conceivable. To display the corresponding point 301 of each image data, a user interface capable of searching each image data independently is disposed. For example, it is possible to turn over and rotate an image by using a rotary encoder.

In step S204, the control circuitry 22, which executes the image registration function 105, converts the coordinates with respect to the second feature value image. First of all, the coordinate conversion is executed with respect to the second feature value image so as to be in the initial positional relationship determined in step S203. Next, for example, the coordinate conversion may be executed based on at least six parameters, namely the rotational movements and translational movements in an X direction, Y direction and Z direction, and, if necessary, based on nine parameters which additionally include three shearing directions.

In step S205, the control circuitry 22, which executes the image registration function 105, checks a coordinate-converted region. Specifically, for example, the control circuitry 22 excludes regions of the feature value image other than the volume data region. The control circuitry 22 may generate, at the same time, an arrangement in which an inside of the region is expressed by “1 (one)” and an outside of the region is expressed by “0 (zero)”.

In step S206, the control circuitry 22, which executes the image registration function 105, calculates an evaluation function relating to displacement as an index for calculating the similarity between the first feature value image and the second feature value image. As the evaluation function, a case of using a correlation coefficient is assumed in the present embodiment, but for example, use may be made of a mutual information and a brightness difference, or general evaluation methods relating to the image registration.

In step S207, the control circuitry 22, which executes the image registration function 105, determines whether or not the evaluation function meets an optimal value reference. If the evaluation function meets the optimal value reference, the process advances to step S209. If the evaluation function fails to meet the optimal value reference, the process advances to step S208. As a method for searching for an optimal positional relationship, a Downhill simplex method and a Powell method are known.

In step S208, for example, the conversion parameter is changed by a Downhill simplex method.

In step S209, the control circuitry 22 determines a displacement amount, and makes a correction by the displacement amount. Thus, the image registration process is completed. The processes in steps S203 and S205 illustrated in FIG. 2 may be omitted as needed.

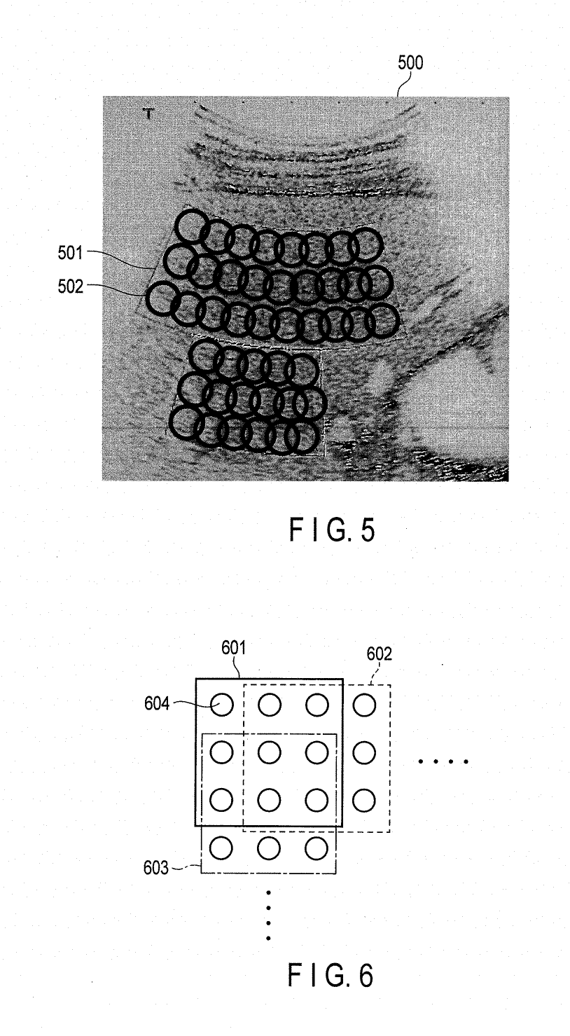

Next, a specific example of a feature value calculation process according to step S201 will be described with reference to FIG. 5.

FIG. 5 is a view illustrating an ultrasonic image 500 to which ROI 501 to be a registration calculation target is set. In the figure, the ultrasonic image is illustrated by black-and-white reverse display. In ROI 501, small regions for calculating a feature value, i.e., small regions 502 for calculating a gradient value of a brightness value, are set. In the present embodiment, it is assumed that the ultrasonic image 500 is an image based on volume data, and thus small regions 502 are actually spheres.

The small region 502 includes a plurality of pixels that form the ultrasonic image 500. The control circuitry 22 calculates a gradient vector of a three-dimensional brightness value at a center of the small region 502 by utilizing the pixels included in the small region, to be set as a feature value. A primary differential of a brightness value I (x, y, z) in a coordinate point (x, y, z) is a vector amount. A gradient vector [symbol] (x,y,z) is described by using a differential in an X direction, Y direction and Z direction.

$${G}_{x} {G}_{x} ( x , y , z ) = \frac{\partial I ( x , y , z )}{\partial x} \frac{\partial I ( x , y , z )}{\partial x}$$

$${G}_{y} {G}_{y} ( x , y , z ) = \frac{\partial I ( x , y , z )}{\partial y} \frac{\partial I ( x , y , z )}{\partial y}$$

$${G}_{z} {G}_{z} ( x , y , z ) = \frac{\partial I ( x , y , z )}{\partial z} \frac{\partial I ( x , y , z )}{\partial z}$$

$$\vec{G} \vec{G} ( x , y , z ) = {G}_{x} {G}_{x} \cdot \vec{{g}_{x}} \vec{{g}_{x}} + {G}_{y} {G}_{y} \cdot \vec{{g}_{y}} \vec{{g}_{y}} + {G}_{z} {G}_{z} \cdot \vec{{g}_{z}} \vec{{g}_{z}}$$

The gradient vector [symbol] (x,y,z) is a primary differential along a direction in which a change rate of a brightness value becomes the largest. A magnitude and a direction of the gradient vector may be a feature value.

The magnitude of the gradient vector can be expressed by the following:

$$‖ \vec{G} ( x , y , z ) ‖ ‖ \vec{G} ( x , y , z ) ‖ = \sqrt{{{G}_{x} ( x , y , z )}^{2} + {{G}_{y } ( x , y , z )}^{2} + {{G}_{z} ( x , y , z )}^{2}} \sqrt{{{G}_{x} ( x , y , z )}^{2} + {{G}_{y } ( x , y , z )}^{2} + {{G}_{z} ( x , y , z )}^{2}}$$

$$or$$

$$‖ \vec{G} ( x , y , z ) ‖ ‖ \vec{G} ( x , y , z ) 〛 = \vert {\overline{G}}_{x} ( x , y , z ) \vert \vert {\overline{G}}_{x} ( x , y , z ) \vert + \vert {G}_{y} ( x , y , z ) \vert + \vert {G}_{z} ( x , y , z ) \vert \vert {G}_{y} ( x , y , z ) \vert + \vert {G}_{z} ( x , y , z ) \vert$$

In addition, it is possible to utilize a secondary differential of a brightness value as a feature value. As a secondary differential, a Laplacian is known.

$$Δ f = \frac{{\partial }^{2} f}{\partial {x}^{2}} Δ f = \frac{{\partial }^{2} f}{\partial {x}^{2}} + \frac{{\partial }^{2} f}{\partial {y}^{2}} \frac{{\partial }^{2} f}{\partial {y}^{2}} + \frac{{\partial }^{2} f}{\partial {z}^{2}} \frac{{\partial }^{2} f}{\partial {z}^{2}}$$

A feature value may be a modification of the above definition by a desired coefficient, etc., utilization of a statistical value in a small region, linear addition of a plurality of values, etc.

A feature value may be a variation in brightness value within a small region. As indices of variation, there are a variance of a brightness value within a small region, a standard deviation, and a relative standard deviation. When a center point in a small region is r, and at a coordinate point i in the small region, a probability distribution of a brightness value of the small region is p(i), an average value is μ, and a variance is σ², a standard deviation (SD) and a relative standard deviation (RSD) are as follows:

$$μ = {\sum }_{i} i \cdot p ( i )$$

$${σ}^{2} = {\sum }_{i} {( i - μ )}^{2} \cdot p ( i )$$

$$S D ( r ) = σ$$

$$R S D ( r ) = \frac{σ}{μ}$$

A feature value may be a modification of the above definition by a desired coefficient, etc.

Furthermore, as a feature value, use may be made of a value obtained by subtracting an average brightness value of a small region from a brightness value, a value obtained by dividing a brightness value of a small region by an average brightness value, or a value obtained by correcting a brightness value of a small region by an average brightness value.

In addition, the small regions 502 may be set so that adjacent small regions 502 do not overlap (so as not to include common pixels), but it is desirable to set the small regions 502 so that adjacent small regions 502 overlap one another (so as to include common pixels). In the example of FIG. 5, the case is assumed in which the small regions 502 are circles (spheres), but the small regions 502 may be rectangles (cubes, rectangular parallelepipeds) or any shape as long as a part of the small region 502 can be appropriately overlapped with adjacent small regions 502.

Specifically, an example of a method of setting small regions will be illustrated in FIG. 6.

As shown in FIG. 6, a case is assumed in which small regions 601, 602, and 603 are rectangles and include four pixels 604 in a shape of 3×3 pixels. The small region 602 adjacent to the small region 601 in the right direction is set to include three pixels on the right column of the small region 601. Similarly, the small region 603 adjacent to the small region 601 in a downward direction is set to include three pixels of the lower half of the small region 601. In each small region, a feature value in the small region may be calculated, and the feature value may be associated with a pixel at a center of the small region. Accordingly, a feature value image having approximately the same number of pixels as that of an ultrasonic image before processing, i.e., a gradient value image, can be generated.

In the above-described example, a process in a two-dimensional ultrasonic image was described, but by processing voxels constituting volume data in the same manner, volume data based on a feature value, i.e., variance volume data, can be generated.

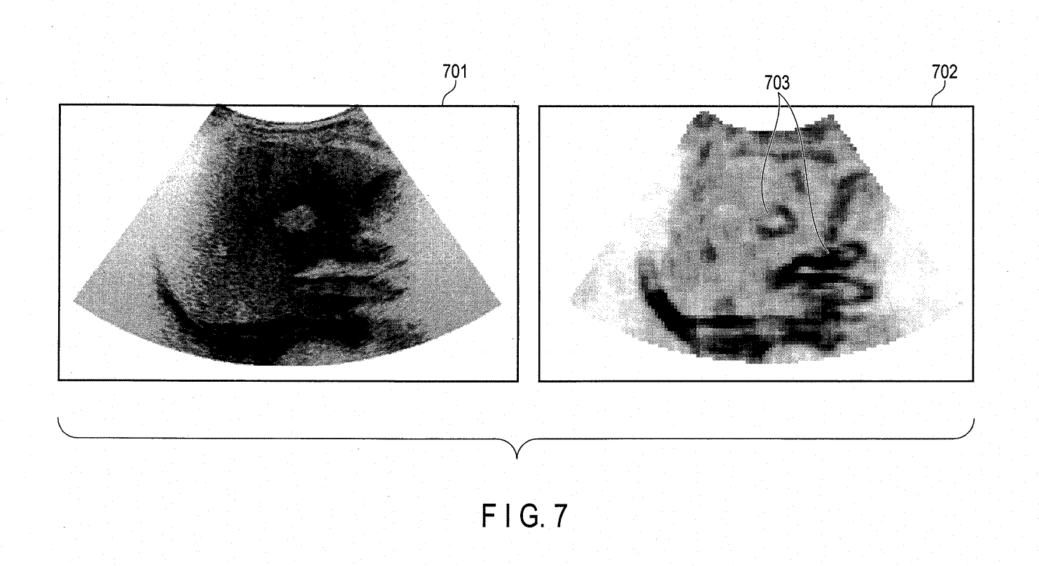

Next, an example of a feature value image generated by the feature value image generation function 103 will be described with reference to FIG. 7.

An image on the left side of FIG. 7 illustrates an ultrasonic image 701 based on volume data upon which a feature value image is based, and an image on the right side illustrates a feature value image 702 generated from the ultrasonic image 701.

When comparing the ultrasonic image 701 and the feature value image 702, portions that can be visually identified as structures in the ultrasonic image 701 are displayed by white regions 703 at the center of the image. This is because the feature value image 702 is an image using the dispersion as a feature value, and differences in variation of brightness distribution in the image are clearly expressed. In both of the ultrasonic image 701 and the feature value image 702, portions indicated by arrows are difficult to identify as to whether they are structures or not by simply visually observing the ultrasonic image 701. However, by generating the feature value image 702, the portions can be easily captured as structures with high precision, and the precision of image registration can be improved.

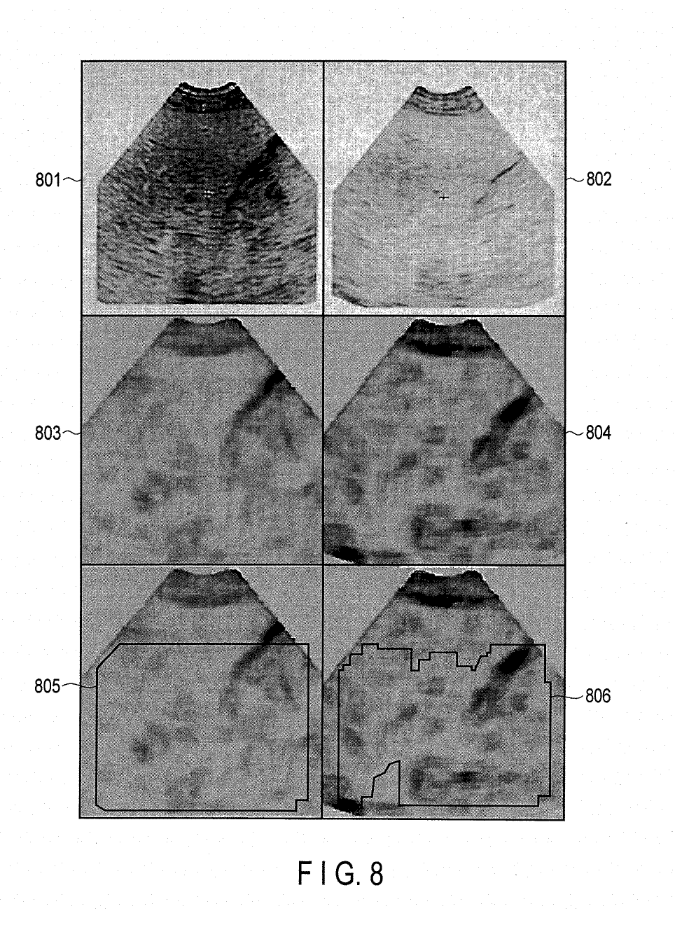

Next, an example of a mask region determined by the region determination function 104 will be described with reference to FIG. 8.

An upper left view of FIG. 8 is a past ultrasonic image (reference ultrasonic image 801), and an upper right view is a current ultrasonic image 802.

An image obtained by subjecting the reference ultrasonic image 801 to the feature value calculation process is a reference feature value image 803, and an image obtained by subjecting the current ultrasonic image 802 to the feature value calculation process is a feature value image 804.

The control circuitry 22, which executes the region determination function 104, sets a mask region 805 as a range (i.e., a range for calculating an evaluation function) for image registration with respect to the reference feature value image 803. The control circuitry 22, which executes the region determination function 104, also sets a mask region 806 as a range for image registration with respect to the feature value image 804.

The image registration function calculates an evaluation function for each of the mask region 805 and the mask region 806 as in step S206, thereby omitting evaluation function calculations for unnecessary regions. Thus, the operation amount in image registration can be reduced, and the precision can be improved. As necessary, image registration may be executed with respect to the entire region, without setting a mask region, of an obtained image.

In the above-described example, a feature value is calculated from a cross-sectional image obtained from volume data, but a feature value may be calculated from B-mode RAW data before being converted into volume data. By calculating a feature value directly from B-mode RAW data without an interpolation process into voxels, the operation amount of data of the feature value calculation process can be reduced.

According to the first embodiment described above, a feature value relating to a gradient vector of brightness and a brightness variation is calculated from medical image data, a feature value image based on the feature value is generated, and image registration between an ultrasonic image and a medical image as a reference is executed by using the feature value image. In this way, by executing image registration by using an image of a feature value, a structure, etc., can be suitably extracted and determined. Thus, it is possible to execute stable image registration with high precision as compared with the conventional methods.

In the first embodiment, registration between the first volume data of ultrasonic image data and the second volume data of past medical image data was described. The case was described in which a pixel value of the ultrasonic image data is a brightness value, but it is possible to execute registration by using a feature value of pixel value distribution of a small region whatever the case may be, in which the pixel value is an ultrasonic echo signal, a Doppler-mode blood flow signal or tissue signal, a strain-mode tissue signal, a ShearWave-mode tissue signal, or a brightness signal of an image.

In addition, image data for registration may exist within ultrasonic image data. Ultrasonic image data has a particular speckle noise, and a structure can be extracted by utilizing a brightness variation of a small region. It is suitable to convert both ultrasonic image data into feature value images and execute registration. As the similarity evaluation function for registration, a cross-correlation, a mutual information, etc., may be utilized. Parameters for extracting the size and a brightness variation of a small region may be common or independent for each ultrasonic image data.

In image registration between ultrasonic image data and CT image data or MR image data, a feature value can be independently defined according to the kind of image. For example, a standard deviation of a small region can be used as a feature value in ultrasonic image data, and the magnitude of a gradient vector can be used as a feature value in CT image data. According to the properties of an image, a feature value and parameters which are excellent in structure extraction can be discretionarily set.

In a case in which a gradient vector is used as a feature value between medical images, it is also possible to normalize by the magnitude of the gradient vector and use the direction of the gradient vector as the feature value. Displacement of the direction of the gradient vector can be used as the similarity evaluation function.

In a case of extracting a feature value of a medical image, a pre-process or post-process may be performed to further clarify a structure. For example, the control circuitry 22 can calculate a feature value relating to a pixel value distribution of a small region after applying a filter process to pixel value data of the medical image as a pre-process. Alternatively, the control circuitry 22 can apply a filter process as a post-process after calculating a feature value relating to a pixel value distribution of a small region and generating a feature value image, thereby further clarifying a structure. As the aforementioned filter, various kinds of filters can be used; for example, a smoothing filter, an anisotropic diffusion filter, and a bilateral filter. In addition, as a post-process, application of a binarization process, etc. is conceivable.

### Second Embodiment

A second embodiment differs from the first embodiment in the point of executing the image registration described in the first embodiment after executing registration (hereinafter, referred to as “sensor registration”) in a sensor coordinate system by using ultrasonic image data acquired by scanning an ultrasonic probe 30 to which position information is added by a position sensor system. Thereby, high-speed and stable image registration can be executed as compared with the first embodiment.

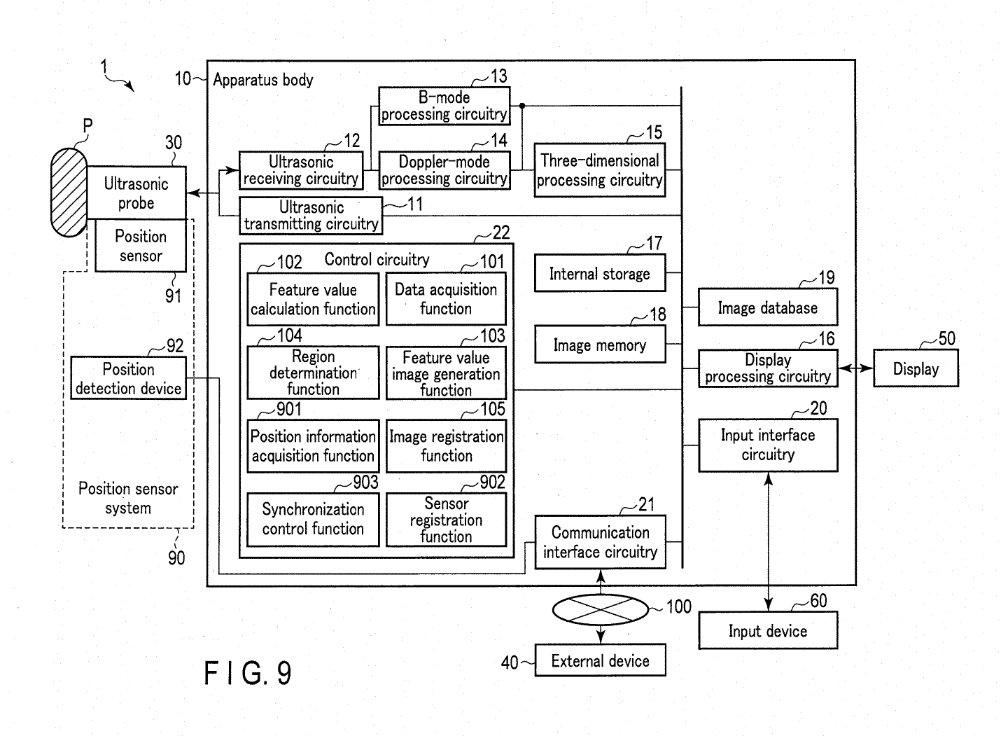

A configuration example of an ultrasonic diagnostic apparatus 1 according to the second embodiment will be described with reference to a block diagram of FIG. 9.

As illustrated in FIG. 9, the ultrasonic diagnostic apparatus 1 includes a position sensor system 90 in addition to the apparatus body 10 and the ultrasonic probe 30 included in the ultrasonic diagnostic apparatus 1 according to the first embodiment.

The position sensor system 90 is a system for acquiring three-dimensional position information of the ultrasonic probe 30 and an ultrasonic image. The position sensor system 90 includes a position sensor 91 and a position detection device 92.

The position sensor system 90 acquires three-dimensional position information of the ultrasonic probe 30 by attaching, for example, a magnetic sensor, an infrared sensor or a target for an infrared camera, as the position sensor 91 to the ultrasonic probe 30. A gyro sensor (angular velocity sensor) may be built in the ultrasonic probe 30, and this gyro sensor may acquire the three-dimensional position information of the ultrasonic probe 30. In addition, the position sensor system 90 may photograph the ultrasonic probe 30 by a camera, and may subject the photographed image to an image recognition process, thereby acquiring the three-dimensional position information of the ultrasonic probe 30. The position sensor system 90 may hold the ultrasonic probe 30 by robotic arms, and may acquire the position of the robotic arms in the three-dimensional space as the position information of the ultrasonic probe 30.

In the description below, a case is described, by way of example, in which the position sensor system 90 acquires position information of the ultrasonic probe 30 by using the magnetic sensor. Specifically, the position sensor system 90 further includes a magnetism generator (not shown) including, for example, a magnetism generating coil. The magnetism generator forms a magnetic field toward the outside, with the magnetism generator itself being set as the center. A magnetic field space, in which position precision is ensured, is defined in the formed magnetic field. Thus, it should suffice if the magnetism generator is disposed such that a living body, which is a target of an ultrasonic examination, is included in the magnetic field space in which position precision is ensured. The position sensor 91, which is attached to the ultrasonic probe 30, detects a strength and a gradient of a three-dimensional magnetic field which is formed by the magnetism generator. Thereby, the position and direction of the ultrasonic probe 30 are acquired. The position sensor 91 outputs the detected strength and gradient of the magnetic field to the position detection device 92.

The position detection device 92 calculates, based on the strength and gradient of the magnetic field which were detected by the position sensor 91, for example, a position of the ultrasonic probe 30 (a position (x, y, z) and a rotational angle (θx, θy, θz) of a scan plane) in a three-dimensional space with the origin set at a predetermined position. At this time, the predetermined position is, for example, a position where the magnetism generator is disposed. The position detection device 92 transmits position information relating to the calculated position (x, y, z, θx, θy, θz) to an apparatus body 10.

In addition to the process according to the first embodiment, a communication interface circuitry 21 is connected to the position sensor system 90, and receives position information which is transmitted from the position detection device 92.

In the meantime, the position information can be imparted to the ultrasonic image data by, for example, three-dimensional processing circuitry 15 associating, by time synchronization, etc., the position information acquired as described above and the ultrasonic image data based on the ultrasonic which is transmitted and received by the ultrasonic probe 30.

When the ultrasonic probe 30, to which the position sensor 91 is attached, is the one-dimensional array probe or 1.5-dimensional array probe, the three-dimensional processing circuitry 15 adds the position information of the ultrasonic probe 30, which is calculated by the position detection device 92, to the B-mode RAW data stored in the RAW data memory. In addition, the three-dimensional processing circuitry 15 may add the position information of the ultrasonic probe 30, which is calculated by the position detection device 92, to the generated two-dimensional image data.

The three-dimensional processing circuitry 15 may add the position information of the ultrasonic probe 30, which is calculated by the position detection device 92, to the volume data. Similarly, when the ultrasonic probe 30, to which the position sensor 91 is attached, is the mechanical four-dimensional probe (three-dimensional probe of the mechanical swing method) or the two-dimensional array probe, the position information is added to the two-dimensional image data.

In addition, control circuitry 22 includes, in addition to each function according to the first embodiment, a position information acquisition function 901, a sensor registration function 902, and a synchronization control function 903.

By executing the position information acquisition function 901, the control circuitry 22 acquires position information relating to the ultrasonic probe 30 from the position sensor system 90 via the communication interface circuitry 21.

By executing the sensor registration function 902, the coordinate system of the position sensor and the coordinate system of the ultrasonic image data are associated. As regards the ultrasonic image data, after the position information is defined by the position sensor coordinate system, the ultrasonic image data with position information are aligned with each other. Between 3D ultrasonic images, the ultrasonic image data is data of a free direction and position, and it is thus necessary to increase the search range for image registration. However, by executing registration in the coordinate system of the position sensor, it is possible to perform rough adjustment of registration between ultrasonic image data. Namely, in the state in which the difference in position and rotation between the ultrasonic image data is decreased, the image registration that is the next step can be executed. In other words, the sensor registration has a function of suppressing the difference in position and rotation between the ultrasonic images within a capture range of an image registration algorithm.

By executing the synchronization control function 903, the control circuitry 22 synchronizes, based on the relationship between a first coordinate system and a second coordinate system, which was determined by the completion of the image registration, a real-time ultrasonic image, which is an image based on ultrasonic image data newly acquired by the ultrasonic probe 30, and a medical image based on medical image data corresponding to the real-time ultrasonic image, and displays the real-time ultrasonic image and the medical image in an interlocking manner.

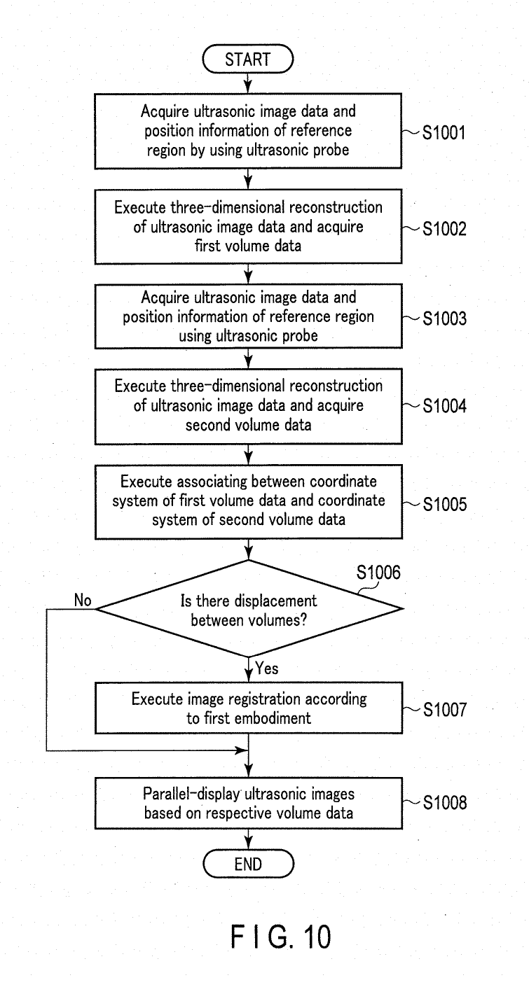

Hereinafter, a description will be given of a registration process of the ultrasonic diagnostic apparatus according to the second embodiment with reference to a flowchart of FIG. 10. In the second embodiment, for example, a case is assumed in which, before the treatment, ultrasonic image data of the vicinity of a living body region (target region) that is the treatment target is acquired, then after the treatment, ultrasonic image data of the treated target region is acquired once again, and the images before and after the treatment are compared, and the effect of the treatment is determined.

In step S1001, the ultrasonic probe 30 of the ultrasonic diagnostic apparatus according to the present embodiment is operated. Thereby, the control circuitry 22, which executes the data acquisition function 101, acquires ultrasonic image data of the target region. In addition, the control circuitry 22, which executes the position information acquisition function 901, acquires the position information of the ultrasonic probe 30 at the time of acquiring the ultrasonic image data from the position sensor system 90, and generates the ultrasonic image data with position information.

In step S1002, the control circuitry 22 or three-dimensional processing circuitry 15 executes three-dimensional reconstruction of the ultrasonic image data by using the ultrasonic image data and the position information of the ultrasonic probe 30, and generates the volume data (first volume data) of the ultrasonic image data with position information. In the meantime, since this ultrasonic image data is ultrasonic image data with position information before the treatment, the ultrasonic image data with position information is stored in an image database 19 as past ultrasonic image data.

Thereafter, a stage is assumed in which the treatment progressed and the operation was finished, and the effect of the treatment is determined.

In step S1003, like step S1001, the control circuitry 22, which executes the position information acquisition function 901 and the data acquisition function 101, acquires the position information of the ultrasonic probe 30 and ultrasonic image data. Like the operation before the treatment, the ultrasonic probe 30 is operated on the target region after the treatment, and the control circuitry 22 acquires the ultrasonic image data of the target region, acquires the position information of the ultrasonic probe 30 from the position sensor system, and generates the ultrasonic image data with position information.

In step S1004, like step S1002, the control circuitry 22 or three-dimensional processing circuitry 15 generates volume data (also referred to as “second volume data”) of the ultrasonic image data with position information, by using the acquired ultrasonic image data and position information.

In step S1005, based on the acquired position information of the ultrasonic probe 30 and ultrasonic image data, the control circuitry 22, which executes the sensor registration function 902, executes sensor registration between the coordinate system (also referred to as “first coordinate system”) of the first volume data and the coordinate system (also referred to as “second coordinate system”) of the second volume data, so that the positions of the target regions may generally match. Both the position of the first volume data and the position of the second volume data are commonly described in the position sensor coordinate system. Accordingly, the registration can directly be executed based on the position information added to volume data.

In step S1006, if the living body does not move during the period from the acquisition of the first volume data to the acquisition of the second volume data, a good registration state can be obtained merely by the sensor registration. In this case, parallel display of ultrasonic images in step S1008 is executed. If a displacement occurs in the sensor coordinate system due to a motion of the body, etc., image registration according to the first embodiment is executed, as step S1007. If the registration result is favorable, parallel display of ultrasonic images in step S1008 is executed.

In step S1008, the control circuitry 22 instructs, for example, display processing circuitry 16 to parallel-display the ultrasonic image before the treatment, which is based on the first volume data, and the ultrasonic image after the treatment, which is based on the second volume data. By the above, the registration process between ultrasonic image data is completed.

In step S1006, even if a displacement does not occur between the volume data, the image registration in step S1007 may by executed.

### (Correction of Displacement Due to Body Motion or Respiratory Time Phase)

During a treatment, in some cases, due to a body motion, a large displacement occurs between ultrasonic image data in the position sensor coordinate system, and this displacement exceeds a correctable range of image registration. There is also a case in which a transmitter of a magnetic field is moved to a position near the patient, from the standpoint of maintaining the magnetic field strength. In such cases, even after the coordinate system of the sensor is associated by the sensor registration function 902, a case is assumed in which a large displacement remains between the ultrasonic image data.

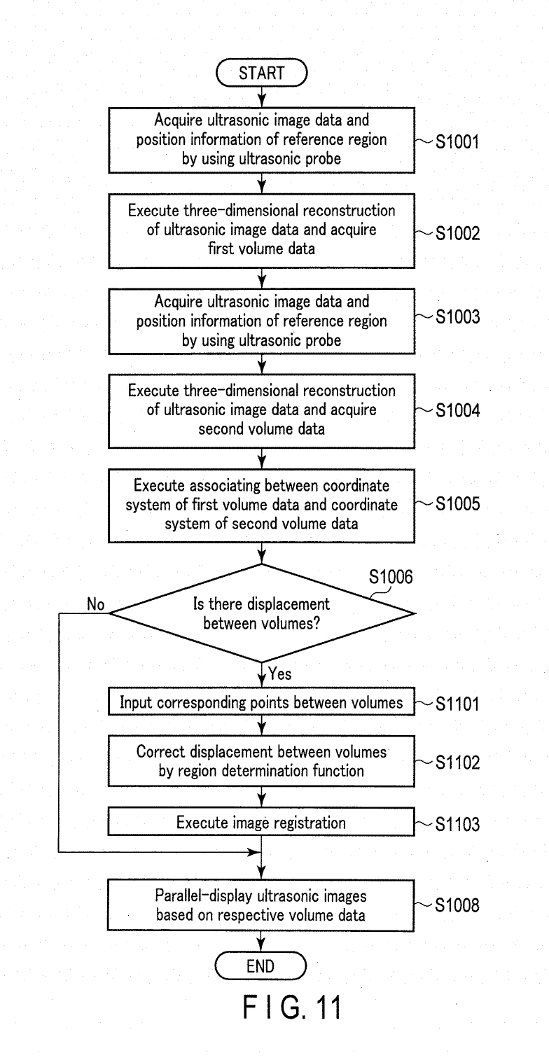

A description will be given of a correction process of displacement with reference to a flowchart of FIG. 11.

The user judges in step S1006 that a large displacement remains even after the sensor registration, and executes a process of step S1101.

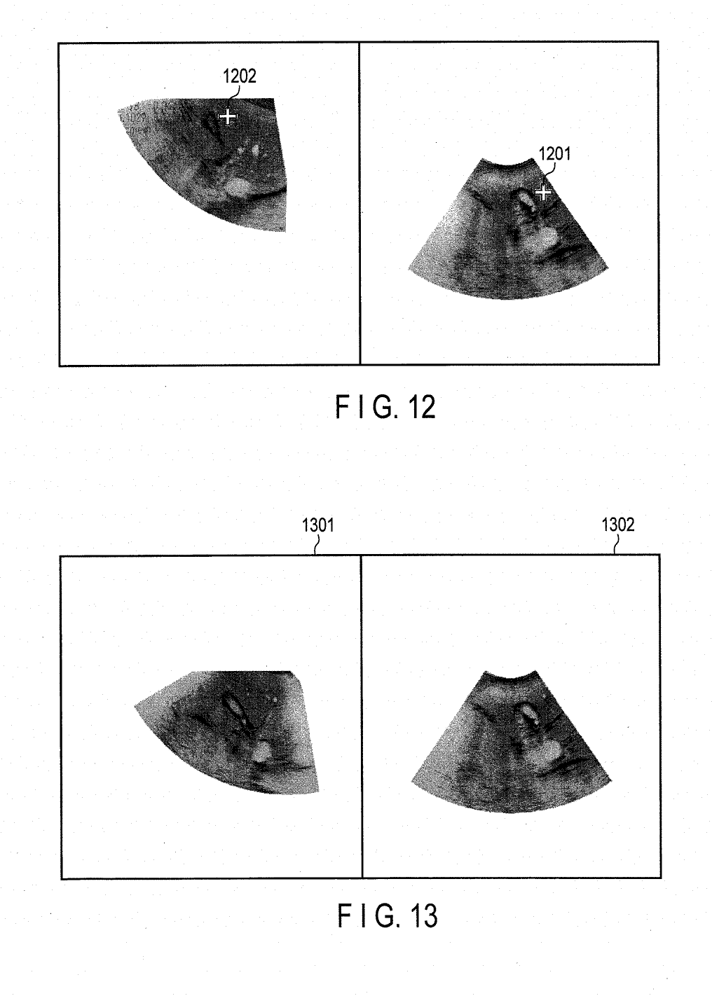

The user designates, in the respective ultrasonic images, corresponding points indicative of a living body region, these points corresponding between the ultrasonic image based on the first volume data and the ultrasonic image based on the second volume data. The method of designating the corresponding points may be, for example, a method in which the user designates the corresponding points by moving a cursor on the screen by using the operation panel through the user interface generated by the display processing circuitry 16, or the user may directly touch the corresponding points on the screen in the case of a touch screen. In an example of FIG. 12, the user designates a corresponding point 1201 on the ultrasonic image based on the first volume data, and designates a corresponding point 1202, which corresponds to the corresponding point 1201, on the ultrasonic image based on the second volume data. The control circuitry 22 displays the designated corresponding points 1201 and 1202, for example, by “+” marks. Thereby, the user can easily understand the corresponding points, and the user can be supported in inputting the corresponding points. The control circuitry 22, which executes the region determination function 104, calculates a displacement between the designated corresponding points 1201 and 1202, and corrects the displacement. The displacement may be corrected, for example, by calculating, as a displacement amount, a relative distance between the corresponding point 1201 and corresponding point 1202, and by moving and rotating, by the displacement amount, the ultrasonic image based on the second volume data.

In the meantime, a region of a predetermined range in the corresponding living body region may be determined as the corresponding region. Also in the case of designating the corresponding region, a similar process as in the case of the corresponding points may be executed.

Furthermore, although the example of correcting the displacement due to the body motion or respiratory time phase has been illustrated, the corresponding points or corresponding regions may be determined in order for the user to designate a region-of-interest (ROI) in the image registration.

After the displacement between the ultrasonic images was corrected by step S1102 of FIG. 11, the user inputs an instruction for image registration, for example, by operating the operation panel or pressing the button attached to the ultrasonic probe 30. In step S1103 of FIG. 11, the control circuitry 22, which executes the image registration function 105, may execute the image registration according to the first embodiment between the ultrasonic image data in which the displacement was corrected.

After the input of the instruction for image registration, the display processing circuitry 16 parallel-displays the ultrasonic images which are aligned in step S1008. Thereby, the user can observe the images by freely varying the positions and directions of the images, for example, by the operation panel of the ultrasonic diagnostic apparatus. In the 3D ultrasonic image data, the positional relationship between the first volume data and second volume data is interlocked, and MPR cross sections can be moved and rotated in synchronism. Where necessary, the synchronization of MPR cross sections can be released, and the MPR cross sections can independently be observed. In place of the operation panel of the ultrasonic diagnostic apparatus, the ultrasonic probe 30 can be used as the user interface for moving and rotating the MPR cross sections. The ultrasonic probe 30 is equipped with a magnetic sensor, and the ultrasonic system can detect the movement amount, rotation amount and direction of the ultrasonic probe 30. By the movement of the ultrasonic probe 30, the positions of the first volume data and second volume data can be synchronized, and the first volume data and second volume data can be moved and rotated.

A display example before image registration between ultrasonic image data is illustrated in FIG. 12.

A left image in FIG. 12 is an ultrasonic image based on the first volume data before the treatment. A right image in FIG. 12 is an ultrasonic image based on the second volume data after the treatment. As illustrated in FIG. 12, if the time of acquisition of ultrasonic image data differs, a displacement may occur due to a body motion, etc., even if the same target region is scanned by the ultrasonic probe 30.

Next, referring to FIG. 13, a description will be given of an example of an ultrasonic image display after the sensor registration and image registration described in the second embodiment.

A left image in FIG. 13 is an ultrasonic image 1301 before the treatment, which is based on the first volume data. A right image in FIG. 13 is an ultrasonic image 1302 after the treatment, which is based on the second volume data. As illustrated in FIG. 13, the ultrasonic image data before and after the treatment are aligned, and the ultrasonic image based on the first volume data is rotated in accordance with the position of the ultrasonic image based on the second volume data, and both images are displayed in parallel. As illustrated in FIG. 13, since the registration between the ultrasonic images is completed, the user can search and display a desired cross section in the aligned state, for example, by a panel operation, and can easily understand the evaluation of the target region (the treatment state of the treatment region).

According to the second embodiment, the sensor registration of the coordinate systems between the ultrasonic image data, which differ with respect to the time of acquisition and the position of acquisition, is executed based on the ultrasonic image data acquired by operating the ultrasonic probe to which the position information is added, and thereafter the image registration is executed. Thereby, the success rate of image registration is increased more than in the first embodiment. This can present to the user a comparison between the ultrasonic images which were easily and exactly aligned.

### Third Embodiment

Although image registration between ultrasonic image data was described in the above-described embodiments, a similar process can be executed in image registration between ultrasonic image data and medical image data other than ultrasonic image data.

Hereinafter, a description will be given of a case of executing registration between a medical image based on medical image data which is obtained by other modalities, such as CT image data, MR image data, X-ray image data and PET image data, and ultrasonic image data which is currently acquired by using an ultrasonic probe 30. In the description below, a case is assumed in which MRI image data is used as the medical image data.

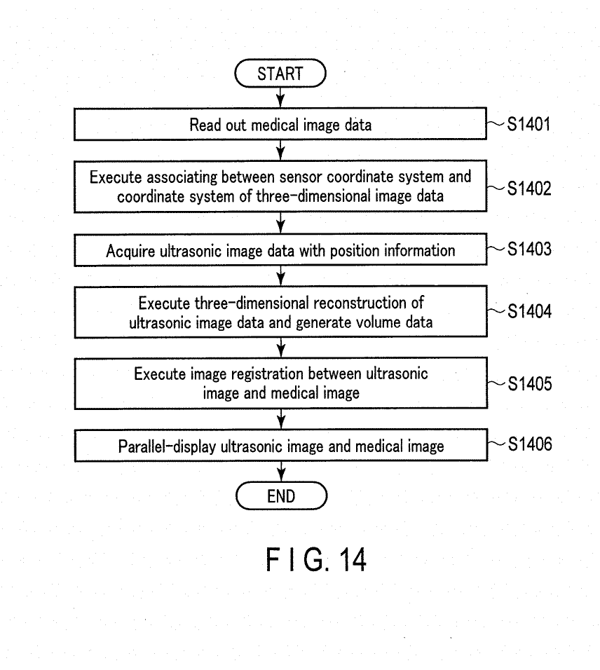

Referring to a flowchart of FIG. 14, a registration process between the ultrasonic image data and the medical image data will be described. Although three-dimensional image data is assumed as the medical image data, two-dimensional image data or four-dimensional image data may be used as the medical image data, as needed.

In step S1401, control circuitry 22 reads out medical image data from an image database 19.

In step S1402, the control circuitry 22 executes associating between the sensor coordinate system of a position sensor system 90 and the coordinate system of the medical image data.

In step S1403, the control circuitry 22, which executes a position information acquisition function 901 and a data acquisition function 101, associates the position information and the ultrasonic image data, which are acquired by the ultrasonic probe 30, thereby acquiring ultrasonic image data with position information.

In step S1404, the control circuitry 22 executes three-dimensional reconstruction of the ultrasonic image data with position information, and generates volume data.

In step S1405, as illustrated in the flowchart of FIG. 2 according to the first embodiment, the control circuitry 22, which executes an image registration function 105, executes image registration between the volume data and the 3D medical image data. In the meantime, generation of a feature value image may be performed with respect to at least ultrasonic image data (volume data), and a feature value image using a feature value of a 3D medical image may be generated as needed.

In step S1406, display processing circuitry 16 parallel-displays the ultrasonic image based on the volume data after the image registration and the medical image based on the 3D medical image data.

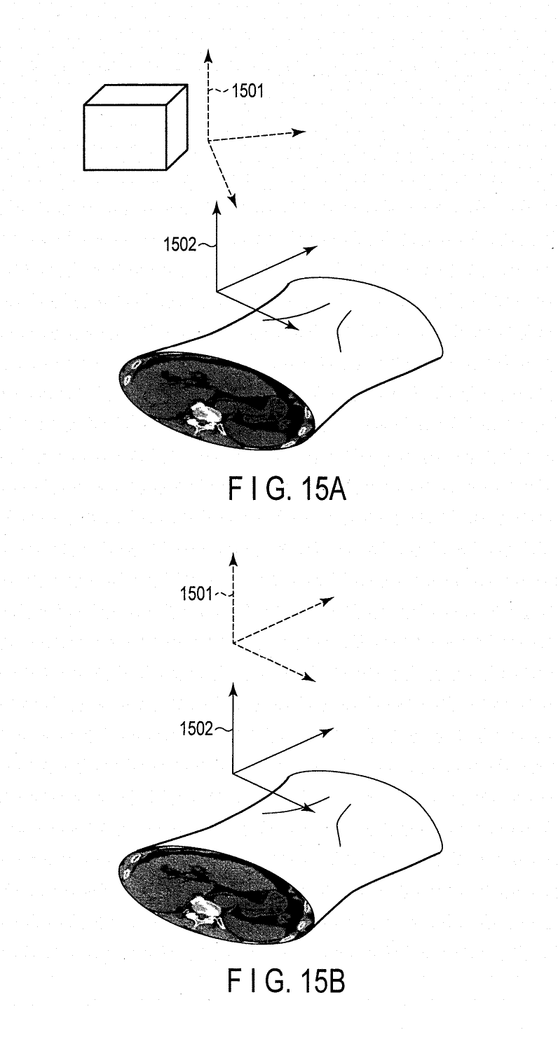

Next, referring to FIG. 15A, FIG. 15B, and FIG. 15C, a description will be given of the associating between the sensor coordinate system and the coordinate system of the 3D medical image data, which is illustrated in step S1402. This associating is a sensor registration process corresponding to step S1006 of the flowchart of FIG. 10.

FIG. 15A illustrates an initial state. As illustrated in FIG. 15A, a position sensor coordinate system 1501 of the position sensor system for generating the position information which is added to the ultrasonic image data, and a medical image coordinate system 1502 of medical image data, are independently defined.

FIG. 15B illustrates a process of registration between the respective coordinate systems. The coordinate axes of the position sensor coordinate system 1501 and the coordinate axes of the medical image coordinate system 1502 are aligned in identical directions. Specifically, the directions of the coordinate axes of the coordinate systems are uniformized.

FIG. 15C illustrates a process of mark registration. FIG. 15C illustrates a case in which the coordinates of the position sensor coordinate system 1501 and the coordinates of the medical image coordinate system 1502 are aligned in accordance with a predetermined reference point. Between the coordinate systems, not only the directions of the axes, but also the positions of the coordinates can be made to match.

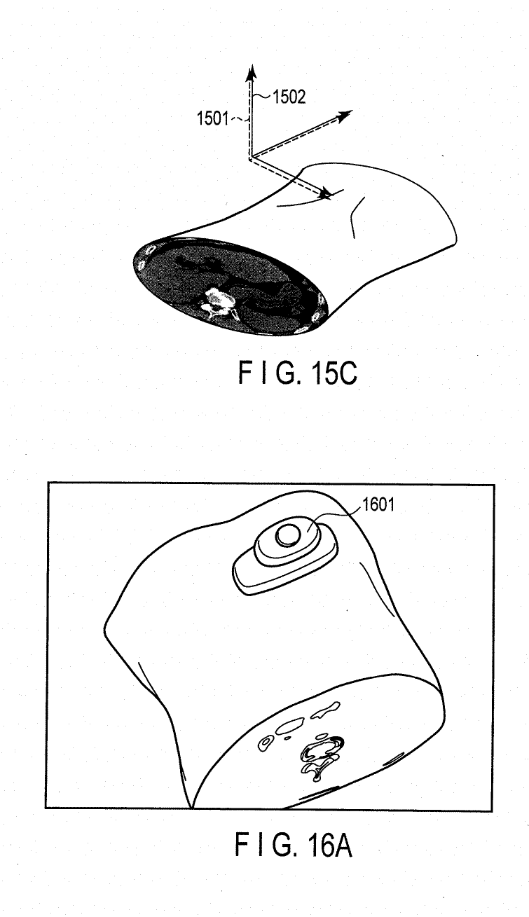

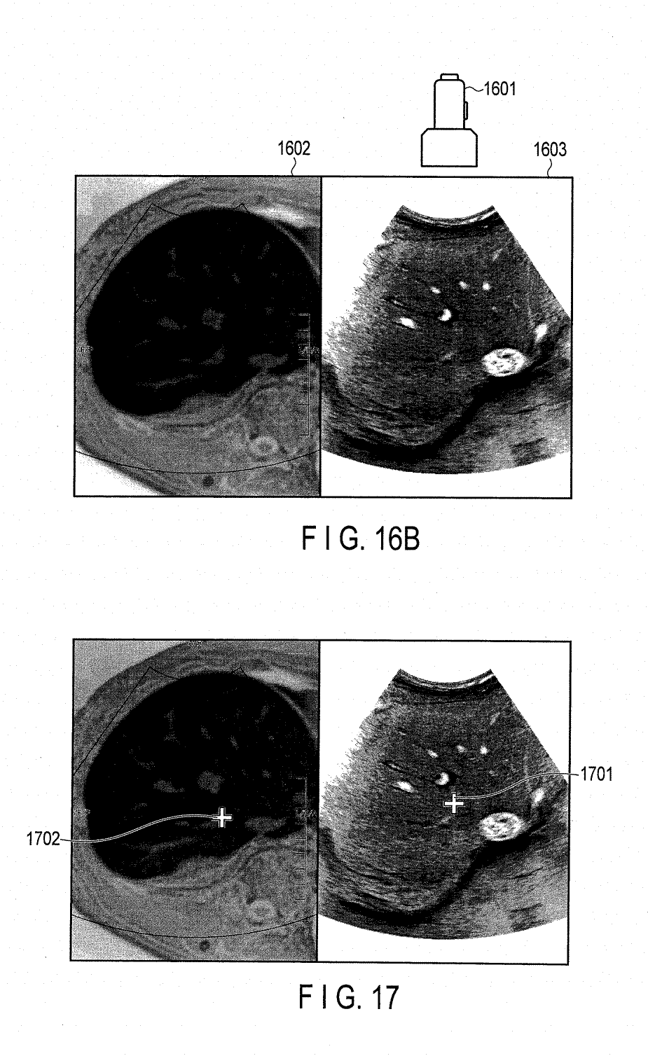

Referring to FIG. 16A and FIG. 16B, a description will be given of a process of realizing, in an actual apparatus, the associating between the sensor coordinate system and the coordinate system of the 3D medical image data.

FIG. 16A is a schematic view illustrating an example of the case in which a doctor performs an examination of the liver. The doctor places the ultrasonic probe 30 horizontally on the abdominal region of the patient. In order to obtain an ultrasonic tomographic image in the same direction as an axial image of CT or MR, the ultrasonic probe 30 is disposed in a direction perpendicular to the body axis, and in such a direction that the ultrasonic tomographic image becomes vertical from the abdominal side toward the back. Thereby, an image as illustrated in FIG. 16B is acquired. In the present embodiment, in step S1401, a three-dimensional MR image is read in from the image database 19, and this three-dimensional MR image is displayed on the left side of the monitor. The MR image of the axial cross section, which is acquired at the position of an icon 1601 of the ultrasonic probe, is an MR image 1602 illustrated in FIG. 16B, and is displayed on the left side of the monitor. Furthermore, a real-time ultrasonic image 1603, which is updated in real time at that time, is displayed on the right side of the monitor in parallel with the MR image 1602. By disposing the ultrasonic probe 30 on the abdominal region as illustrated in FIG. 16A, the ultrasonic tomographic image in the same direction as the axial plane of the MR can be acquired.

The user puts the ultrasonic probe 30 on the body surface of the living body in the direction of the axial cross section. The user confirms, by visual observation, whether or not the ultrasonic probe 30 is in the direction of the axial cross section. When the user puts the ultrasonic probe 30 on the living body in the direction of the axial cross section, the user performs a registration process such as clicking by the operation panel, or pressing of the button. Thereby, the control circuitry 22 acquires and associates the sensor coordinates of the position information of the sensor of the ultrasonic probe 30 in this state, and the MR image data coordinates of the position of the MPR plane of the MR image data. The axial cross section in the MR image data of the living body can be converted to the position sensor coordinates, and can be recognized. Thereby, the registration (matching of directions of coordinate axes of coordinate systems) illustrated in FIG. 16B is completed. In the registration state, the system can associate the MPR image of the MR and the real-time ultrasonic tomographic image by the sensor coordinates, and can display these images in an interlocking manner. At this time, since the axes of both coordinate systems are coincident, the directions of the images match, but a displacement remains in the position of the body axis direction. By moving the ultrasonic probe 30 in the state in which the displacement remains in the position of the body axis direction, the user can observe the MPR plane of the MR and the real-time ultrasonic image in an interlocking manner.

Next, referring to FIG. 17, a description will be given of the method of realizing, by the apparatus, the process of the mark registration illustrated in FIG. 15C.

FIG. 17 illustrates a parallel-display screen of the MR image 1602 and real-time ultrasonic image 1603 illustrated in FIG. 16B, the parallel-display screen being displayed on the monitor.

After the completion of the registration, by moving the ultrasonic probe 30 in the state in which the displacement remains in the position of the body axis direction, the user can observe the MPR plane of the MR and the real-time ultrasonic image in an interlocking manner.

While viewing the real-time ultrasonic image 1603 which is displayed on the monitor, the user scans the ultrasonic probe 30, thereby causing the monitor to display a target region (or an ROI) such as the center of the region for registration or a structure. Thereafter, the user designates the target region as a corresponding point 1701 by the operation panel, etc. In the example of FIG. 17, the designated corresponding point is indicated by “+”. At this time, the system acquires and stores the position information of the sensor coordinate system of the corresponding point 1701.

Next, the user moves the MPR cross section of the MR by moving the ultrasonic probe 30, and displays the cross-sectional image of the MR image, which corresponds to the cross section including the corresponding point 1701 of the ultrasonic image designated by the user. When the cross-sectional image of the MR image, which corresponds to the cross section including the corresponding point 1701, was displayed, the user designates a target region (or an ROI), such as the center of the region for registration or a structure, which is designated on the cross-sectional image of the MR image, as a corresponding point 1702 by the operation panel, etc. At this time, the system acquires and stores the position information of the coordinate system of the MR image data of the corresponding point 1702.

The control circuitry 22, which executes a region determination function 104, corrects a displacement between the coordinate system of the MR image data and the sensor coordinate system, based on the position of the designated corresponding point in the sensor coordinate system and the position of the designated corresponding point in the coordinate system of the MR image data. Specifically, for example, based on a difference between the corresponding point 1701 and corresponding point 1702, the control circuitry 22 corrects a displacement between the coordinate system of the MR image data and the sensor coordinate system, and aligns the coordinate systems. Thereby, the process of mark registration of FIG. 15C is completed, and the step S1402 of the flowchart of FIG. 14 is finished.

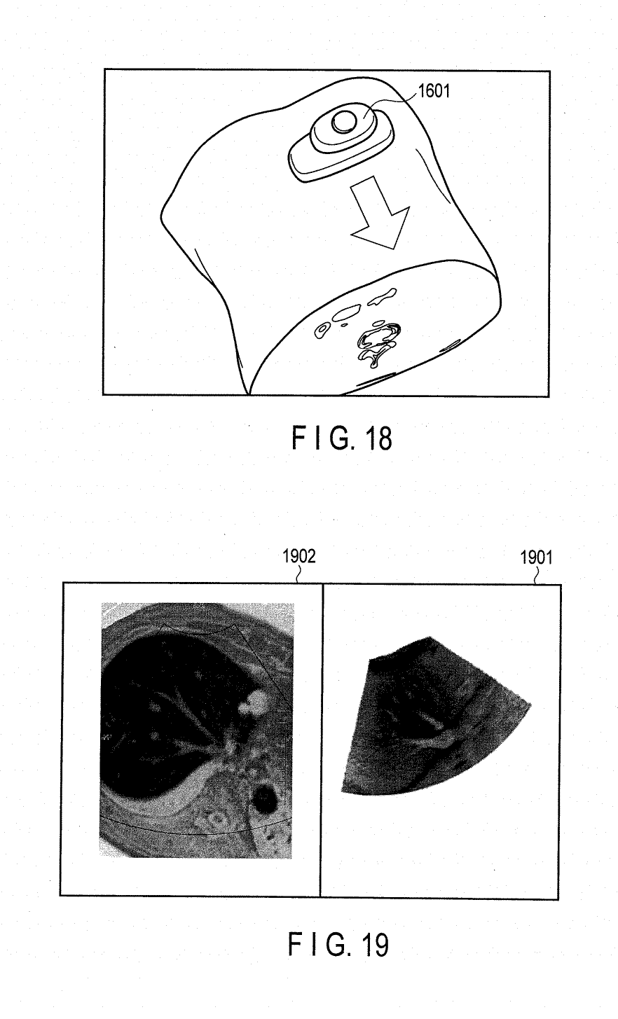

Next, referring to a schematic view of FIG. 18, a description will be given of an example of acquisition of ultrasonic image data in the step S1403 of the flowchart of FIG. 14, in the state in which the coordinate system of the MR image data and the sensor coordinate system are aligned.

After the completion of the position correction, the user manually operates the ultrasonic probe 30 with respect to the region including the target region, while referring to the three-dimensional MR image data, and acquires the ultrasonic image data with position information. Next, the user presses the switch for image registration, and executes image registration. By the process thus far, the position of the MR image data and the position of the ultrasonic image data are made to generally match, and the MR image data and the ultrasonic image data include the common target. Thus, the operation of image registration is well performed.

An example of the ultrasonic image display after the image registration will be described with reference to FIG. 19. As in the step S1406 of FIG. 14, the ultrasonic image, which is aligned with the MR image, is parallel-displayed.

As illustrated in FIG. 19, an ultrasonic image 1901 of ultrasonic image data is rotated and displayed in accordance with the image registration, so as to correspond to an MR 3D image 1902 of MR 3D image data. Thus, it becomes easier to understand the positional relationship between the ultrasonic image and MR 3D image. It is possible to observe the image by freely changing the position and direction of the image by the operation panel, etc. of the ultrasonic diagnostic apparatus. The positional relationship between the MR 3D image data and the 3D ultrasonic image data is interlocked, and the MPR cross sections can be synchronously moved and rotated. Where necessary, the synchronization of MPR cross sections can be released, and the MPR cross sections can independently be observed. In place of the operation panel of the ultrasonic diagnostic apparatus, the ultrasonic probe 30 can be used as the user interface for moving and rotating the MPR cross sections. The ultrasonic probe 30 is equipped with the magnetic sensor, and the ultrasonic system can detect the movement amount, rotation amount and direction of the ultrasonic probe 30. By the movement of the ultrasonic probe 30, the positions of the MR 3D image data and the 3D ultrasonic image data can be synchronized, and can be moved and rotated.

In the third embodiment, the MR 3D image data was described by way of example. However, the third embodiment is similarly applicable to other 3D medical image data of CT, X-ray, ultrasonic, PET, etc. The associating between the coordinate system of 3D medical image data and the coordinate system of the position sensor was described in the steps of registration and mark registration illustrated in FIG. 15A, FIG. 15B and FIG. 15C. However, the registration between the coordinates is possible by various methods. It is possible to adopt some other methods, such as a method of executing registration by designating three or more points in both coordinate systems. Besides, instead of acquiring the ultrasonic image data with position information after the completion of the correction of displacement, it is possible to acquire the ultrasonic image data with position information before the completion of the correction of displacement, to generate the volume data, to designate the corresponding points between the ultrasonic image based on the volume data of the ultrasonic image data and the medical image based on the 3D medical image data, and to correct the displacement.

### (Synchronous Display Between Ultrasonic Image and Medical Image)

If the above-described sensor registration and image registration are completed, the relationship between the coordinate system of the medical image (the MR coordinate system in this example) and the position sensor coordinate system is determined. The display processing circuitry 16 refers to the position information of the real-time (live) ultrasonic image acquired by the user freely moving the ultrasonic probe 30 after the completion of the registration process, and can thereby display the MPR cross section of the corresponding MR. The corresponding cross sections of the highly precisely aligned MR image and real-time ultrasonic image can be interlock-displayed (also referred to as “synchronous display”).

Synchronous display can also be executed between 3D ultrasonic images by the same method. Specifically, a 3D ultrasonic image, which was acquired in the past, and a real-time 3D ultrasonic image can be synchronously displayed. In the step S1008 of FIG. 10 and FIG. 11 and the step S1406 of FIG. 14, the parallel synchronous display of the 3D medical image and the aligned 3D ultrasonic image was illustrated. However, by utilizing the sensor coordinates, the real-time ultrasonic tomographic image can be switched and displayed.

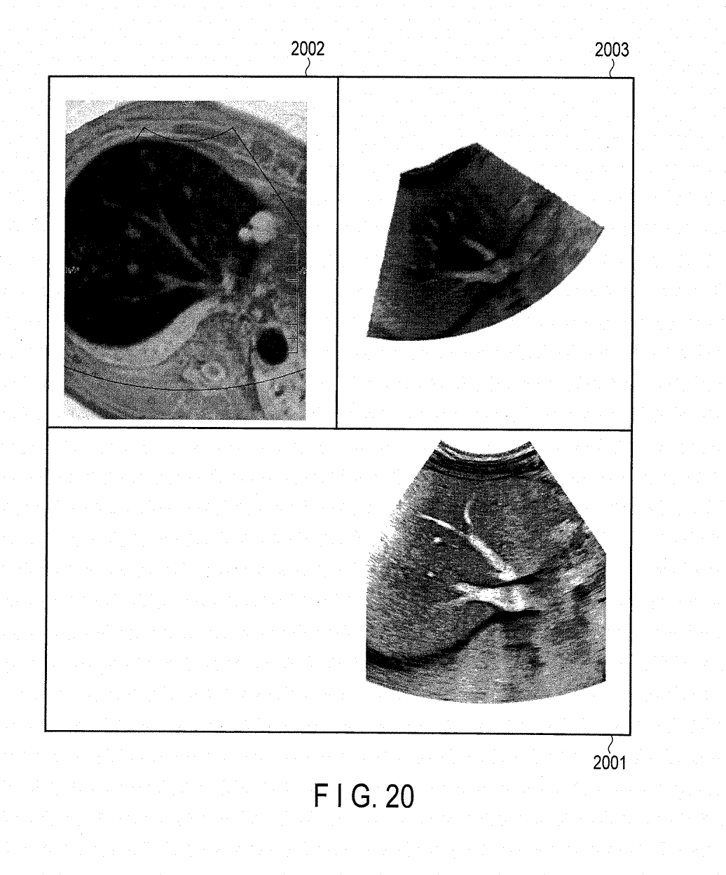

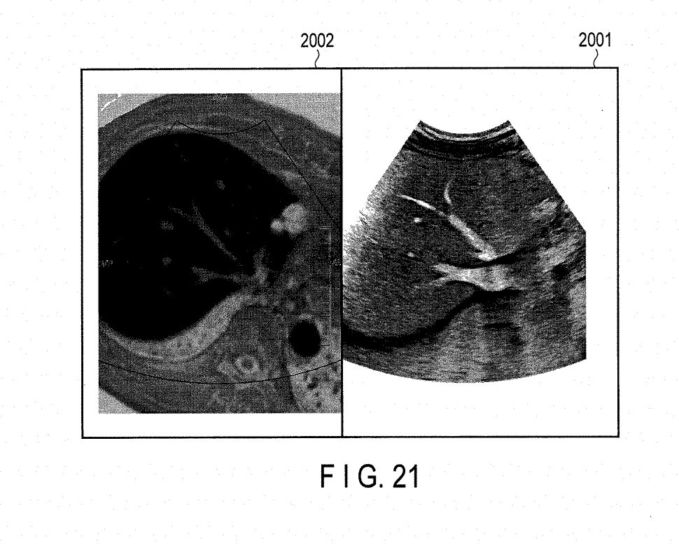

FIG. 20 illustrates an example of synchronous display of the ultrasonic image and medical image. For example, if the ultrasonic probe 30 is scanned, a real-time ultrasonic image 2001, a corresponding MR 3D image 2002, and an ultrasonic image 2003 for registration, which was used for registration, are displayed. In the meantime, as illustrated in FIG. 21, the real-time ultrasonic image 2001 and MR 3D image 2002 may be parallel-displayed, without displaying the ultrasonic image 2003 for registration.

Although it is presupposed that sensor registration is executed between ultrasonic image data and medical image data in the third embodiment, only image registration may be executed, without executing the sensor registration. When executing image registration, it is desirable to calculate a feature value and generate a feature value image at least with respect to ultrasonic image data. As for medical image data, on the other hand, a structure of a living body is more distinctive than that in an ultrasonic image, and thus a feature value image may or may not be generated.

According to the third embodiment described above, by executing image registration by using a value in a mask region of a feature value image based on a feature value, not original volume data, the image registration between an ultrasonic image and a medical image based on medical image data other than ultrasonic image can also be executed with high precision.

Thus, the ultrasonic image and medical image, which were easily and exactly aligned, can be presented to the user. In addition, since the sensor coordinate system and the coordinate system of the medical image, for which the image registration is completed, are synchronized, the MPR cross section of the 3D medical image and real-time ultrasonic tomographic image can be synchronously displayed in interlock with the scan of the ultrasonic probe 30. Specifically, the exact comparison between the medical image and ultrasonic image can be realized, and the objectivity of ultrasonic diagnosis can be improved.

In the above-described embodiments, the position sensor systems, which utilize magnetic sensors, have been described.

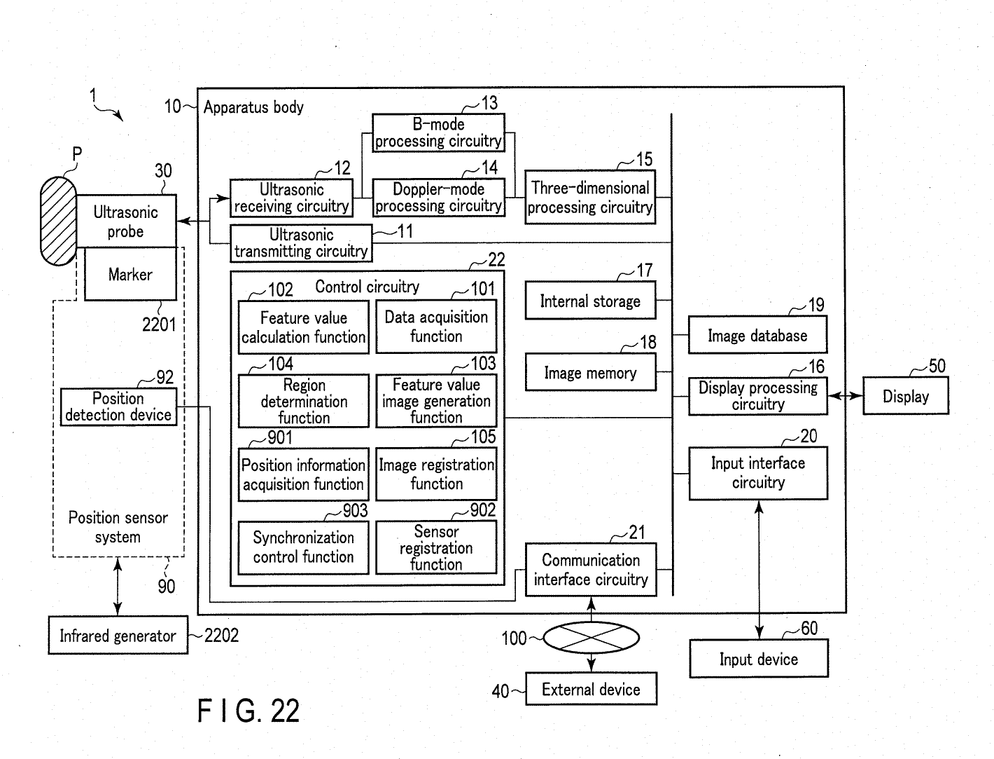

FIG. 22 illustrates an embodiment in a case in which infrared is utilized in the position sensor system. Infrared is transmitted at least in two directions by an infrared generator 2202. The infrared is reflected by a marker 2201 which is disposed on the ultrasonic probe 30. The infrared generator 2202 receives the reflected infrared, and the data is transmitted to the position sensor system 90. The position sensor system 90 detects the position and direction of the marker from the infrared information observed from plural directions, and transmits the position information to the ultrasonic diagnostic apparatus.

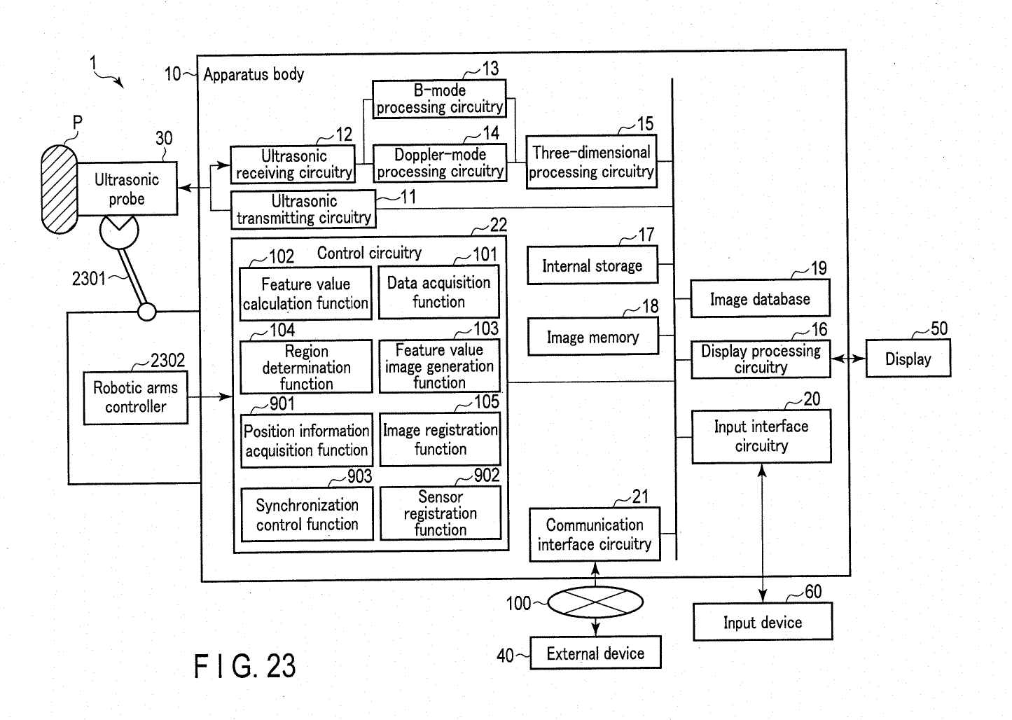

FIG. 23 illustrates an embodiment in a case in which robotic arms are utilized in the position sensor system. Robotic arms 2301 move the ultrasonic probe 30. Alternatively, the doctor moves the ultrasonic probe 30 in the state in which the robotic arms 2301 are attached to the ultrasonic probe 30. A position sensor is attached to the robotic arms 2301, and position information of each part of the robotic arms is successively transmitted to a robotic arms controller 2302. The robotic arms controller 2302 converts the position information to position information of the ultrasonic probe 30, and transmits the converted position information to the ultrasonic diagnostic apparatus.

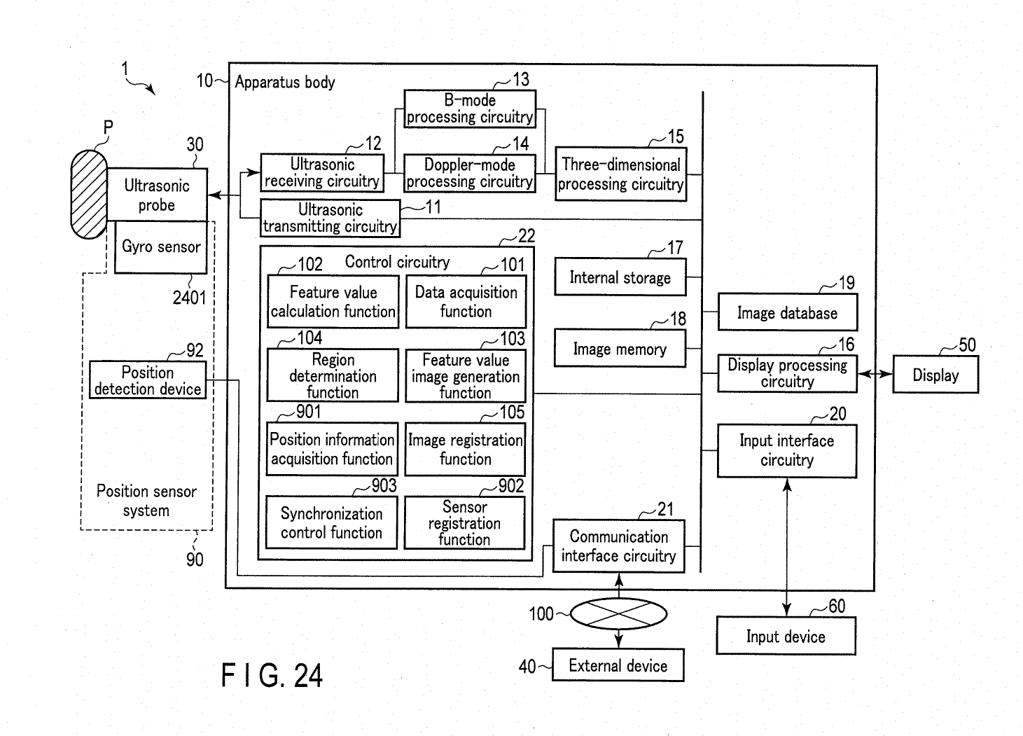

FIG. 24 illustrates an embodiment in a case in which a gyro sensor is utilized in the position sensor system. A gyro sensor 2401 is built in the ultrasonic probe 30, or is disposed on the surface of the ultrasonic probe 30. Position information is transmitted from the gyro sensor 2401 to the position sensor system 90 via a cable. In some cases, as the cable, a part of a cable for the ultrasonic probe 30 may be used, or a dedicated cable may be used. In addition, the position sensor system 90 may be a dedicated unit in some cases, or the position sensor system 90 may be realized by software in the ultrasonic apparatus in other cases. The gyro sensor can integrate an acceleration or rotation information with respect to a predetermined initial position, and can detect changes in position and direction. It can be thought that the position is corrected by GPS information. Alternatively, by an input of the user, initial position setting or correction can be executed. By the position sensor system 90, the information of the gyro sensor is converted to position information by an integration process, etc., and the converted position information is transmitted to the ultrasonic diagnostic apparatus.

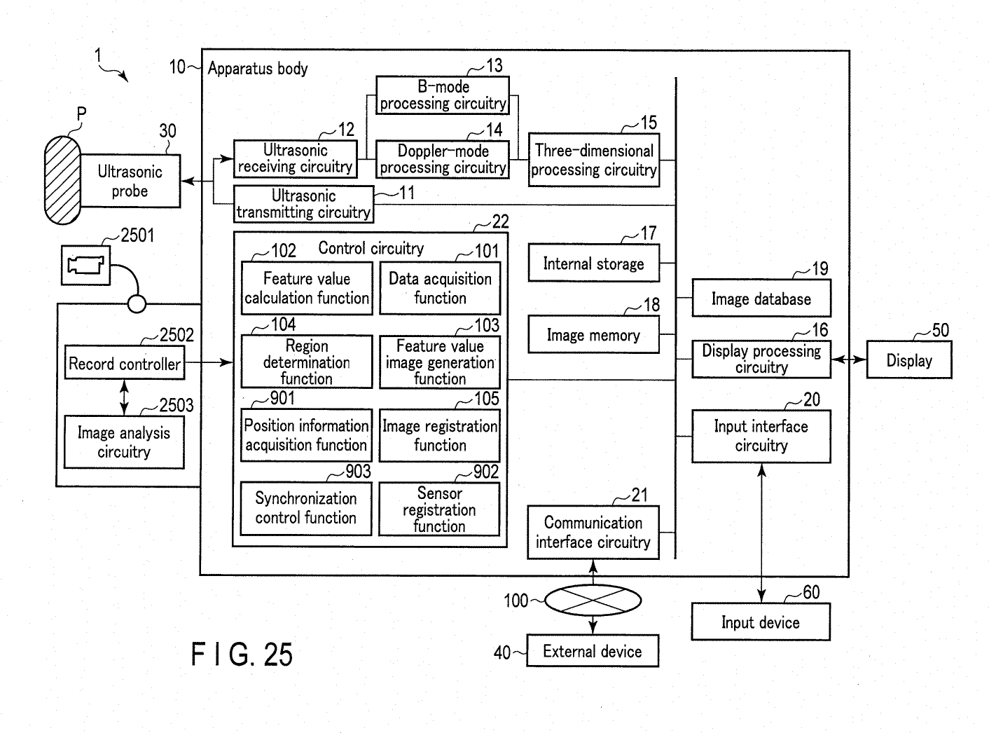

FIG. 25 illustrates an embodiment in a case in which a camera is utilized in the position sensor system. The vicinity of the ultrasonic probe 30 is photographed by a camera 2501 from a plurality of directions. The photographed image is sent to image analysis circuitry 2503, and the ultrasonic probe 30 is automatically recognized and the position is calculated. A record controller 2502 transmits the calculated position to the ultrasonic diagnostic apparatus as position information of the ultrasonic probe 30.

The term “processor” used in the above description means, for example, a CPU (Central Processing Unit), a GPU (Graphics Processing Unit), or circuitry such as an ASIC (Application Specific Integrated Circuit), or a programmable logic device (e.g. SPLD (Simple Programmable Logic Device) and CPLD (Complex Programmable Logic Device)), and FPGA (Field Programmable Gate Array). The processor realizes functions by reading out and executing programs stored in the storage circuitry. In the meantime, each processor of the embodiments is not limited to the configuration in which each processor is configured as single circuitry. Each processor of the embodiments may be configured as a single processor by combining a plurality of independent circuitries, thereby to realize the function of the processor. Furthermore, a plurality of structural elements in FIG. 1 may be integrated into a single processor, thereby to realize the function of the processor. In addition, an image diagnostic apparatus including each processor described above in the present embodiment can be operated.

In the above description, the case is assumed in which ultrasonic image data and medical image data for registration are between two data, but the case is not limited thereto. Registration may be executed among three or more data; for example, ultrasonic image data currently acquired by scanning an ultrasonic probe and two or more ultrasonic image data which were photographed in the past, and the respective data may be parallel-displayed. Alternatively, registration may be executed among currently-scanned ultrasonic image data, and one or more ultrasonic image data and one or more three-dimensional CT image data which were photographed in the past, and the respective data may be parallel-displayed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
processing circuitry configured to:
set a plurality of small regions in at least one of a plurality of medical image data;
calculate a feature value of pixel value distribution of each small region;
generate a feature value image of the at least one of the plurality of medical image by using the calculated feature value; and
execute an image registration between the plurality of medical image data by utilizing the feature value image.

2. The apparatus according to claim 1, wherein the feature value is a value relating to a pixel value variation of the small region.

3. The apparatus according to claim 2, wherein the feature value is a standard deviation or a variance.

4. The apparatus according to claim 2, wherein the feature value is a value obtained by subtracting an average brightness of the small region from a pixel value of each pixel of the small region.

5. The apparatus according to claim 1, wherein the feature value is a value relating to a primary differential of a pixel value of the small region.

6. The apparatus according to claim 5, wherein the feature value is a gradient vector or a gradient value.

7. The apparatus according to claim 1, wherein the feature value is a feature value relating to a secondary differential of a pixel value of the small region.

8. The apparatus according to claim 7, wherein the feature value is a Laplacian of a pixel value.

9. The apparatus according to claim 1, wherein the at least one of the plurality of medical image data is ultrasonic image data, and a pixel value is a value obtained from any one of an ultrasonic echo signal, a Doppler-mode blood flow signal, a Doppler-mode tissue signal, a strain-mode tissue signal, a ShearWave-mode tissue signal, and a brightness signal of an image.

10. The apparatus according to claim 1, wherein the at least one of the plurality of medical image data is three-dimensional data obtained by using any one of ultrasound, a computed tomography (CT), a magnetic resonance (MR), X-ray, and a positron emission tomography (PET).

11. The apparatus according to claim 1, wherein the at least one of the plurality of medical image data is subjected to a smoothing filter process, a bilateral filter process, or an anisotropic diffusion filter process before the feature value is calculated.

12. The apparatus according to claim 1, wherein the feature value image is subjected to a smoothing filter process, a bilateral filter process, an anisotropic diffusion filter process, or a binarization process after the feature value image is generated.

13. The apparatus according to claim 1, wherein the processing circuitry utilizes a cross-correlation or mutual information for similarity evaluation of images.

14. The apparatus according to claim 6, wherein the gradient vector is normalized by amplitude.

15. The apparatus according to claim 1, wherein the processing circuitry utilizes an inner product and an outer product of a gradient vector for similarity evaluation of images.

16. The apparatus according to claim 1, wherein in each of the plurality of medical image data for the image registration, a feature value of pixel value distribution of each small region or accompanying parameters can be independently set.

17. The apparatus according to claim 1, wherein the processing circuitry is further configured to determine an initial positional relationship for registration between the plurality of medical image data.

18. An ultrasonic diagnostic apparatus comprising:
processing circuitry configured to:
acquire position information relating to an ultrasonic probe and an ultrasonic image;
acquire ultrasonic image data which is obtained by a transmission and reception of ultrasonic waves from the ultrasonic probe at a position where the position information is acquired, the ultrasonic image data being associated with the position information;
execute associating between a first coordinate system of ultrasonic image data relating to the position information and a second coordinate system relating to medical image data;
set a plurality of small regions in at least one of the associated the ultrasonic image data and the medical image data;
calculate a feature value of pixel value distribution of each small region;
generate a feature value image by using the feature value; and
execute an image registration between image data by utilizing the feature value image.

19. The apparatus according to claim 18, wherein the medical image data is ultrasonic image data.

20. A medical image diagnostic assistance method comprising:
setting a plurality of small regions in at least one of a plurality of medical image data;
calculating a feature value of pixel value distribution of each small region;
generating a feature value image of the at least one of the plurality of medical image by using the calculated feature value; and
executing an image registration between the plurality of medical image data by utilizing the feature value image.

21. The method according to claim 20, further comprising determining an initial positional relationship for registration between the plurality of medical image data.

22. A medical image diagnostic assistance method comprising:
acquiring position information relating to an ultrasonic probe and an ultrasonic image;
acquiring ultrasonic image data which is obtained by a transmission and reception of ultrasonic waves from the ultrasonic probe at a position where the position information is acquired, the ultrasonic image data being associated with the position information;
executing associating between a first coordinate system of ultrasonic image data relating to the position information and a second coordinate system relating to medical image data;
setting a plurality of small regions in at least one of the associated the ultrasonic image data and the medical image data;
calculating a feature value of pixel value distribution of each small region;
generating a feature value image by using the feature value; and
executing an image registration between image data by utilizing the feature value image.

23. A medical image diagnostic assistance method comprising:
acquiring stored position information relating to medical image data;
executing associating between a first coordinate system of ultrasonic image data relating to the stored position information and a second coordinate system of medical image data;
setting a plurality of small regions in at least one of the associated ultrasonic image data and medical image data and calculating a feature value of pixel value distribution of each small region;
generating a feature value image by using the feature value; and
executing an image registration between image data by utilizing the feature value image.
